(19) United States
(12) Patent Application Publication
(10) Pub. No.: US 2004/0082757 A1
(43) Pub. Date: Apr. 29, 2004

(54) Echinocandin derivatives, pharmaceutical compositions containing same and use thereof as drugs

(76) Inventors: Katsuhiko Ito, Osaka (JP); Yutaka Nishihara, Toyama (JP); Mitsutaka Matsurura, Osaka (JP); Kazumi Kawai, Osaka (JP); Masayoshi Kinoshita, Osaka (JP); Tomoji Higaki, Osaka (JP); Shigehiro Takase, Osaka (JP); Hidenori Ohki, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/468,142

(22) PCT Filed: Feb. 25, 2002
(86) PCT No.: PCT/JP02/01677

(30) Foreign Application Priority Data
Feb. 26, 2001 (AU) PR3364
Feb. 26, 2001 (AU) PR3363

(51) Int. Cl. C07K 7/64

(52) U.S. Cl. 530/317

(57) ABSTRACT

This invention relates to new polypeptide compound represented by the following formula (I), wherein R¹, R², R³, R⁴, R⁵, R⁶, and R⁷ are described in the specification, or a salt thereof which have antimicrobial activities (especially, antifungal activities), inhibitory activity on β-1,3-glucan synthase, to process for preparation thereof, to a pharmaceutical composition comprising the same, and to a method for the prophylactic and/or therapeutic treatment of infectious diseases including Pneumocystis carinii infection (e.g. Pneumocystis carinii pneumonia) in a human being or an animal.

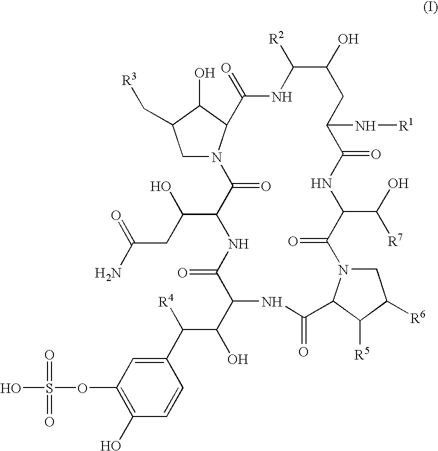

### TECHNICAL FIELD

The present invention relates to new polypeptide compound or a salt thereof which are useful as a medicament.

### BACKGROUND ART

In U.S. Pat. No. 5,376,634, U.S. Pat. No. 5,502,033, etc., there are disclosed the polypeptide compound or a salt thereof, which have antimicrobial activities (especially antifungal activity).

### DISCLOSURE OF INVENTION

The present invention relates to new polypeptide compound or a salt thereof.

More particularly, it relates to new polypeptide compound or a salt thereof, which have antimicrobial activities [especially, antifungal activities, in which the fungi may include Aspergillus, Cryptococcus, Candida, Mucor, Actinomyces, Histoplasma, Dermatophyte, Malassezia, Fusarium and the like.], inhibitory activity on β-1,3-glucan synthase, and further which are expected to be useful for the prophylactic and/or therapeutic treatment of Pneumocystis carinii infection (e.g. Pneumocystis carinii pneumonia) in a human being or an animal, to a process for preparation thereof, to a pharmaceutical composition comprising the same, and to a method for the prophylactic and/or therapeutic treatment of infectious diseases including Pneumocystis carinii infection (e.g. Pneumocystis carinii pneumonia) in a human being or an animal.

The object polypeptide compound of the present invention is new and can be represented by the following general formula (I):

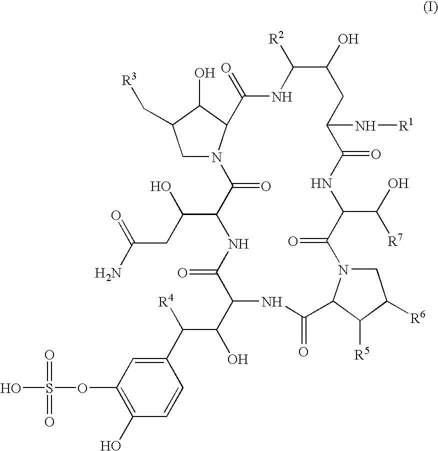

wherein R¹ is hydrogen or acyl group,

R², R³, R⁴, R⁵ and R⁶ are each independently hydrogen or hydroxy, and

R⁷ is hydrogen or lower alkyl,

or a salt thereof.

The polypeptide compound (I) of the present invention can be prepared by the processes as illustrated in the following schemes.

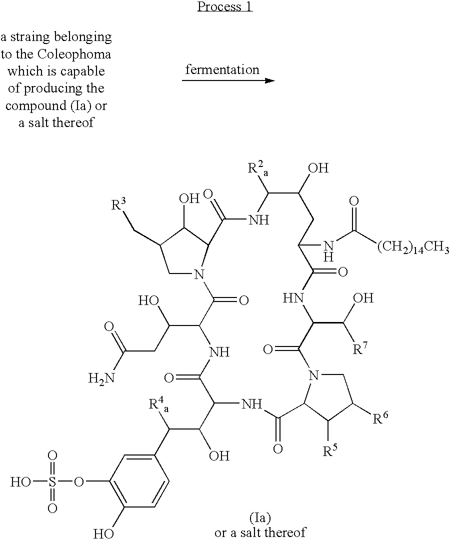

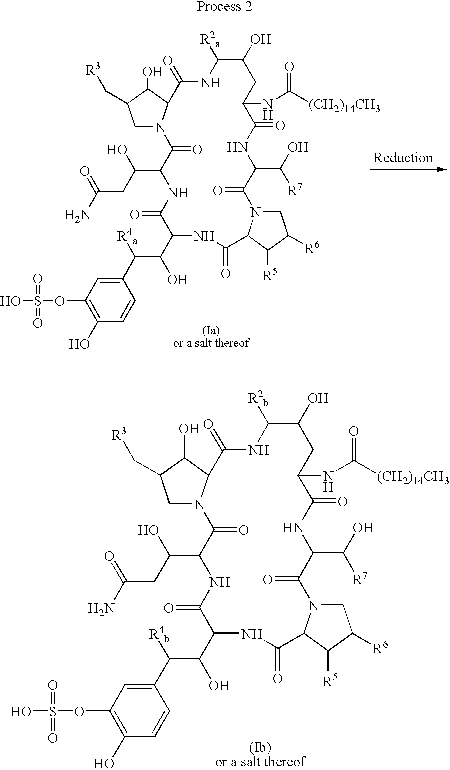

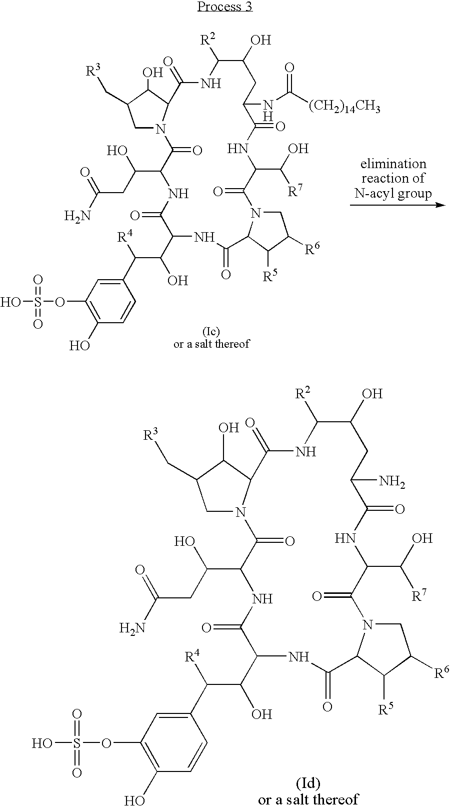

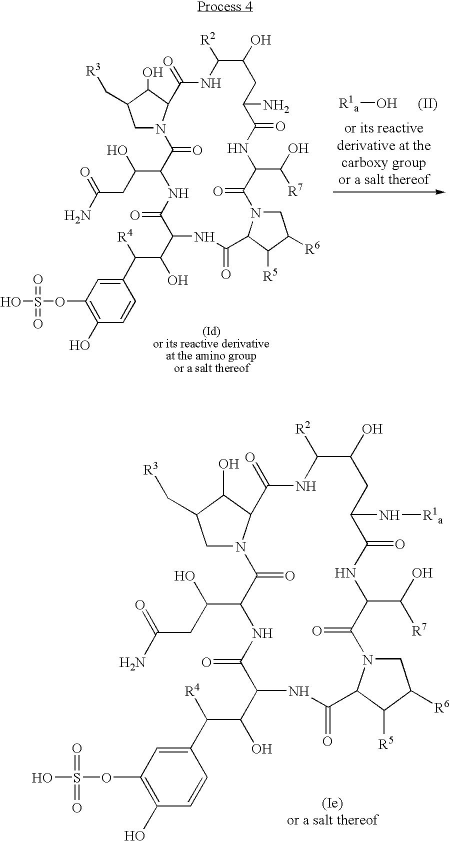

wherein R², R³, R⁴, R⁵, R⁶ and R⁷ are defined above,

Rₐ² is acyl group,

Rₐ² and Rₐ⁴ are hydroxy, and

R_{b}² and R_{b}⁴ are hydrogen.

Suitable salts of the compound (I), (Ia), (Ib), (Ic), (Id) and (Ie) are pharmaceutically acceptable and conventional non-toxic mono or di salt(s) and include a metal salt such as an alkali metal salt [e.g., sodium salt, potassium salt, etc.] and an alkaline earth metal salt [e.g., calcium salt, magnesium salt, etc.], an ammonium salt, an organic base salt [e.g. trimethylamine salt, triethylamine salt, N,N′-diisopropylethylamine salt, pyridine salt, picoline salt, dicyclohexylamine salt, N,N-dibenzylethylenediamine salt, diisopropylethylamine salt, etc.], an organic acid addition salt [e.g. formate, acetate, trifluoroacetate, maleate, tartrate, methanesulfonate, benzenesulfonate, toluenesulfonate, etc.], an inorganic acid addition salt [e.g. hydrochloride, hydrobromide, hydroiodide, sulfate, phosphate, etc.], a salt with an amino acid [e.g. arginine salt, aspartic acid salt, glutamic acid salt, etc.], and the like.

In the above and subsequent descriptions of the present specification, suitable examples and illustration of the various definitions which the present invention intends to include within the scope thereof are explained in detail as follows.

The term “lower” is intended to mean 1 to 6 carbon atom(s), unless otherwise indicated.

The term “higher” is intended to mean 7 to 20 carbon atoms, unless otherwise indicated.

The term “one or more” may be the number of 1 to 6, unless otherwise indicated.

Suitable example of “halogen” may be fluoro, chloro, bromo, iodo, and the like, unless otherwise indicated.

Suitable example of “lower alkoxy” may include straight or branched one such as methoxy, ethoxy, propoxy, isopropoxy, butoxy, isobutoxy, tert-butoxy, pentyloxy, tert-pentyloxy, neo-pentyloxy, hexyloxy, isohexyloxy, and the like, in which the preferred one may be propoxy, pentyloxy and hexyloxy.

Suitable example of “higher alkoxy” may include straight or branched one such as heptyloxy, octyloxy, 3,5-dimethyloctyloxy, 3,7-dimethyloctyloxy, nonyloxy, decyloxy, undecyloxy, dodecyloxy, tridecyloxy, tetradecyloxy, hexadecyloxy, heptadecyloxy, octadecyloxy, nonadecyloxy, icosyloxy, and the like, in which the preferred one may be (C₇-C₁₄)alkoxy, and the most preferred one may be octyloxy.

Suitable example of “lower alkyl” may include straight or branched one having 1 to 6 carbon atom(s), such as methyl, ethyl, propyl, isopropyl, butyl, isobutyl, sec-butyl, tertbutyl, pentyl, tert-pentyl, neo-pentyl, hexyl, isohexyl and the like, unless otherwise indicated.

Suitable example of “higher alkyl” may include straight or branched one having 7 to 20 carbon atoms, such as heptyl, octyl, 3,5-dimethyloctyl, 3,7-dimethyloctyl, nonyl, decyl, undecyl, dodecyl, tridecyl, tetradecyl, pentadecyl, hexadecyl, heptadecyl, octadecyl, nonadecyl, icosyl, and the like, unless otherwise indicated.

Suitable example of “aryl” and “ar” moiety may include phenyl which may have lower alkyl (e.g., phenyl, mesityl, tolyl, etc.), naphthyl, anthryl, and the like, in which the preferred one may be phenyl.

Suitable example of “aroyl” may include benzoyl, toluoyl, naphthoyl, anthrylcarbonyl, and the like, in which the preferred one may be benzoyl.

Suitable example of “heterocyclic group” and “heterocyclic” moiety may include

unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), etc.;

saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, etc.;

unsaturated condensed heterocyclic group containing 1 to 4 nitrogen atom(s), for example, indolyl, isoindolyl, indolinyl, indolizinyl, benzimidazolyl, quinolyl, isoquinolyl, indazolyl, benzotriazolyl, etc.;

unsaturated 3 to 8-membered (more preferably 5 or 6-membered) heteromonocyclic group containing 1 or 2 oxygen atom(s) and 1 to 3 nitrogen atom(s), for example, oxazolyl, isoxazolyl, oxadiazolyl (e.g., 1,2,4-oxadiazolyl, 1,3,4-oxadiazolyl, 1,2,5-oxadiazolyl, etc.), etc.;

saturated 3 to 8-membered (more preferably 5 or 6-membered) heteromonocyclic group containing 1 or 2 oxygen atom(s) and 1 to 3 nitrogen atom(s), for example, morpholinyl, sydnonyl, etc.;

unsaturated condensed heterocyclic group containing 1 or 2 oxygen atom(s) and 1 to 3 nitrogen atom(s), for example, benzoxazolyl, benzoxadiazolyl, etc.;

unsaturated 3 to 8-membered (more preferably 5 or 6-membered) heteromonocyclic group containing 1 or 2 sulfur atom(s) and 1 to 3 nitrogen atom(s), for example, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;

saturated 3 to 8-membered (more preferably 5 or 6-membered) heteromonocyclic group containing 1 or 2 sulfur atom(s) and 1 to 3 nitrogen atom(s), for example, thiazolidinyl, etc.;

unsaturated 3 to 8-membered (more preferably 5 or 6-membered) heteromonocyclic group containing 1 or 2 sulfur atom(s), for example, thienyl, dihydrodithiinyl, dihydrodithionyl, etc.;

unsaturated condensed heterocyclic group containing 1 or 2 sulfur atom(s) and 1 to 3 nitrogen atom(s), for example, benzothiazolyl, benzothiadiazolyl, imidazothiadiazolyl, etc.;

unsaturated 3 to 8-membered (more preferably 5 or 6-membered) heteromonocyclic group containing an oxygen atom, for example, furyl, etc.;

saturated 3 to 8-membered (more preferably 5 or 6-membered) heteromonocyclic group containing an oxygen atom, for example, tetrahydrofuran, tetrahydropyran, etc.;

unsaturated 3 to 8-membered (more preferably 5 or 6-membered) heteromonocyclic group containing an oxygen atom and 1 or 2 sulfur atom(s), for example, dihydrooxathiinyl, etc.;

unsaturated condensed heterocyclic group containing 1 or 2 sulfur atom(s), for example, benzothienyl, benzodithiinyl, etc.;

unsaturated condensed heterocyclic group containing an oxygen atom and 1 or 2 sulfur atom(s), for example, benzoxathiinyl, etc.; and the like.

Suitable example of “acyl group” may include aliphatic acyl, aromatic acyl, heterocyclic acyl, arylaliphatic acyl and heterocyclic-aliphatic acyl derived from carboxylic acid, carbonic acid, carbamic acid, sulfonic acid, and the like.

Suitable example of “acyl group” thus explained may be carboxy; carbamoyl; mono or di(lower)alkylcarbamoyl (e.g., methylcarbamoyl, dimethylcarbamoyl, ethylcarbamoyl, diethylcarbamoyl, etc.);

Aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.);

lower or higher alkoxycarbonyl (e.g., methoxycarbonyl, ethoxycarbonyl, t-butoxycarbonyl, t-pentyloxycarbonyl, heptyloxycarbonyl, etc.); lower alkenyloxycarbonyl (e.g., vinyloxycarbonyl, propenyloxycarbonyl, allyloxycarbonyl, butenyloxycarbonyl, butedienyloxycarbonyl, pentenyloxycarbonyl, hexenyloxycarbonyl, etc.);

lower or higher alkylsulfonyl (e.g., methylsulfonyl, ethylsulfonyl, etc.);

lower or higher alkoxysulfonyl (e.g., methoxysulfonyl, ethoxysulfonyl, etc.); or the like;

Aromatic acyl such as aroyl (e.g., benzoyl, toluoyl, naphthoyl, etc.);

ar(lower)alkanoyl [e.g., phenyl(C₁-C₆)alkanoyl (e.g., phenylacetyl, phenylpropanoyl, phenylbutanoyl, phenylisobutanoyl, phenylpentanoyl, phenylhexanoyl, etc.), naphthyl(C₁-C₆)alkanoyl (e.g., naphthylacetyl, naphthylpropanoyl, naphthylbutanoyl, etc.), etc.];

ar(lower)alkenoyl (e.g., phenyl(C₃-C₆)alkenoyl (e.g., phenylpropenoyl, phenylbutenoyl, phenylmethacryloyl, phenylpentanoyl, phenylhexanoyl, etc.), naphthyl(C₃-C₆)alkenoyl (e.g., naphthylpropenoyl, naphthylbutenoyl, etc.), etc.];

ar(lower)alkoxycarbonyl [e.g., phenyl(C₁-C₆)alkoxycarbonyl (e.g., benzyloxycarbonyl, etc.), fluorenyl(C₁-C₆)alkoxycarbonyl (e.g., fluorenylmethyloxycarbonyl, etc.), etc.];

aryloxycarbonyl (e.g., phenoxycarbonyl, naphthyloxycarbonyl, etc.);

aryloxy(lower)alkanoyl (e.g., phenoxyacetyl, phenoxypropionyl, etc.);

arylcarbamoyl (e.g., phenylcarbamoyl, etc.);

arylthiocarbamoyl (e.g., phenylthiocarbamoyl, etc.);

arylglyoxyloyl (e.g., phenylglyoxyloyl, naphthylglyoxyloyl, etc.);

arylsulfonyl which may have 1 to 4 lower alkyl (e.g., phenylsulfonyl, p-tolylsulfonyl, etc.);

aroyl (e.g., benzoyl) substituted with one or more suitable substituent(s); or the like;

Heterocyclic acyl such as

heterocycliccarbonyl;

heterocyclic(lower)alkanoyl (e.g., heterocyclicacetyl, heterocyclicpropanoyl, heterocyclicbutanoyl, heterocyclicpentanoyl, heterocyclichexanoyl, etc.);

heterocyclic(lower)alkenoyl (e.g., heterocyclicpropenoyl, heterocyclicbutenoyl, heterocyclicpentenoyl, heterocyclichexenoyl, etc.);

heterocyclicglyoxyloyl; or the like; in which suitable “heterocyclic” moiety in the terms “heterocycliccarbonyl”, “heterocyclic(lower)alkanoyl”, “heterocyclic(lower)alkenoyl” and “heterocyclicglyoxyloyl” can be referred to aforementioned “heterocyclic” moiety.

Suitable example of “acyl group” of R¹ can be referred to aforementioned “acyl group”, in which the preferred one may be lower alkoxycarbonyl, higher alkanoyl, and aroyl substituted with heterocyclic group which may have one or more suitable substituent(s).

Suitable example of “aroyl” moiety in the term of “aroyl substituted with heterocyclic group which may have one or more suitable substituent(s)” can be referred to aforementioned “aroyl”, in which the preferred one may be benzoyl.

Suitable example of “heterocyclic group” moiety in the term of “aroyl substituted with heterocyclic group which may have one or more suitable substituent(s)” can be referred to aforementioned “heterocyclic group”, in which the preferred one may be saturated 3 to 8-membered heteromonocyclic group containing 1 to 4 nitrogen atom(s), unsaturated 3 to 8-membered heteromonocyclic group containing 1 or 2 sulfur atom(s) and 1 to 3 nitrogen atom(s), unsaturated 3 to 8-membered heteromonocyclic group containing 1 or 2 oxygen atom(s) and 1 to 3 nitrogen atom(s) and unsaturated condensed heterocyclic group containing 1 or 2 sulfur atom(s) and 1 to 3 nitrogen atom(s) and the more preferred one may be piperazinyl, thiadiazolyl, oxadiazolyl, imidazothiadiazolyl and isoxazolyl.

Suitable example of “suitable substituent(s)” moiety in the term of “aroyl substituted with heterocyclic group which may have one or more suitable substituent(s)” can be referred to aforementioned “suitable substituent(s)”, in which the preferred one may be aryl which has one or more higher alkoxy, aryl which has one or more lower alkoxy, aryl which has one or more cyclo(lower)alkyl, aryl which has one or more lower alkoxy(higher)alkoxy, aryl which has one or more heterocyclic groups, cyclo(lower)alkyl which may have one or more cyclo(lower)alkyl, aryl substituted with aryl which may have one or more lower alkoxy, aryl substituted with aryl which may have one or more higher alkoxy, aryl substituted with aryl which may have one or more lower alkoxy having heterocyclic group, aryl which has one or more lower alkoxy(lower)alkoxy, heterocyclic group which may have one or more higher alkyl, aryl substituted with aryl which may have one or more aryloxy(lower)alkoxy, aryl substituted with aryl which may have one or more lower alkenyloxy, aryl substituted with aryl which may have one or more lower alkoxy(higher)alkoxy, aryl substituted with aryl which has one or more heterocyclic(lower)alkoxy, in which heterocyclic group may have one or more lower alkyl, aryl which has one or more aryloxy(lower)alkoxy, heterocyclic group which may have one or more heterocyclic groups, aryl which has one or more cyclo(lower)alkyloxy, aryl which has one or more heterocyclic groups having lower alkoxy, aryl which has one or more heterocyclic groups having cyclo(lower)alkyloxy, aryl which has one or more heterocyclic groups having aryl(lower)alkyloxy, aryl which has one or more heterocyclic groups having cyclo(lower)alkyl, aryl which has one or more heterocyclic groups having aryl, heterocyclic group which may have one or more aryl having lower alkoxy, heterocyclic group which may have one or more aryl having higher alkoxy(lower)alkyl, heterocyclic group which may have one or more aryl having lower alkoxy(lower)alkoxy, heterocyclic group which may have one or more aryl having cyclo(lower)alkyl, heterocyclic group which may have one or more aryl having heterocyclic group, heterocyclic group which may have one or more aryl substituted with heterocyclic(lower)alkyl having aryl, heterocyclic group which may have one or more heterocyclic groups having aryl, aryl substituted with aryl which may have one or more cyclo(lower)alkyloxy, aryl substituted with aryl which may have one or more lower alkoxy(lower)alkyl, aryl substituted with aryl which may have one or more lower alkoxy(lower)alkoxy, aryl substituted with aryl which may have one or more lower alkoxy(lower)alkoxy(lower)alkyl, aryl substituted with aryl which may have one or more lower alkoxy(lower)alkoxy(lower)alkoxy, aryl substituted with aryl which may have one or more heterocyclic groups, aryl which has one or more cyclo(lower)alkyloxy, aryl which has one or more lower alkoxy(higher)alkylthio, aryl which has one or more lower alkoxy having heterocyclic group, cyclo(lower)alkyl which may have one or more lower alkyl, cyclo(lower)alkyl which may have one or more aryl, aryl,

in which the preferred one may be phenyl having (C₇-C₁₄)alkoxy, phenyl having (C₄-C₆)alkoxy, phenyl having cyclo(C₄-C₆)alkyl, phenyl having (Cₗ-C₄)alkoxy(C₇-C₁₄)alkoxy, phenyl having saturated 3 to 8-membered heteromonocyclic group containing 1 to 4 nitrogen atom(s), cyclo(C₄-C₆)alkyl having cyclo(C₄-C₆)alkyl, phenyl substituted with phenyl having (C₁-C₆)alkoxy, phenyl substituted with phenyl having (C₇-C₁₄)alkoxy, phenyl substituted with phenyl which has (C₁-C₄)alkoxy having saturated 3 to 8-membered heteromonocyclic group containing 1 to 4 nitrogen atom(s), phenyl having (C₁-C₄)alkoxy(C₄-C₆)alkoxy, unsaturated 3 to 8-membered heteromonocyclic group containing 1 to 4 nitrogen atom(s) having (C₇-C₁₄)alkyl, phenyl substituted with phenyl having phenyloxy(C₁-C₄)alkoxy, phenyl substituted with phenyl having (C₃-C₆)alkenyloxy, phenyl substituted with phenyl having (C₁-C₄)alkoxy(C₇-C₁₄)alkoxy, phenyl substituted with phenyl which has (C₁-C₄)alkoxy having saturated 3 to 8-membered heteromonocylic group containing 1 or 2 oxygen atom(s) and 1 to 3 nitrogen atom(s) having 1 to 4 (C₁-C₄)alkyl, phenyl having phenyloxy(C₁-C₄)alkoxy, phenyl having (C₁-C₄)alkoxy(C₇-C₁₄)alkoxy, unsaturated 3 to 8-membered heteromonocyclic group containing 1 to 4 nitrogen atom(s) having saturated 3 to 8-membered heteromonocyclic group containing 1 to 4 nitrogen atom(s), phenyl having cyclo(C₄-C₆)alkyloxy, phenyl which has saturated 3 to 8-membered heteromonocyclic group containing 1 to 4 nitrogen atom(s) having (C₁-C₄)alkoxy, phenyl which has saturated 3 to 8-membered heteromonocyclic group containing 1 to 4 nitrogen atom(s) having cyclo(C₄-C₆)alkyloxy, phenyl which has saturated 3 to 8-membered heteromonocyclic group containing 1 to 4 nitrogen atom(s) having phenyl(C₁-C₄)alkyloxy, phenyl which has saturated 3 to 8-membered heteromonocyclic group containing 1 to 4 nitrogen atom(s) having cyclo(C₄-C₆)alkyl, phenyl which has saturated 3 to 8-membered heteromonocyclic group containing 1 to 4 nitrogen atom(s) substituted with cyclo(C₄-C₆)alkyl having di(C₁-C₄)alkyl, phenyl which has. saturated 3 to 8-membered heteromonocyclic group containing 1 to 4 nitrogen atom(s) substituted with cyclo(C₄-C₆)alkyl having (C₁-C₄)alkyl, phenyl which has saturated 3 to 8-membered heteromonocyclic group containing 1 to 4 nitrogen atom(s) substituted with (C₁-C₄)alkoxy and phenyl having halogen, phenyl which has saturated 3 to 8-membered heteromonocyclic group containing 1 to 4 nitrogen atom(s) substituted with phenyl, phenyl which has unsaturated 3 to 8-membered heteromonocyclic group containing 1 or 2 sulfur atom(s) and 1 to 3 nitrogen atom(s) substituted with phenyl having (C₁-C₆)alkoxy, unsaturated 3 to 8-membered heteromonocyclic group containing 1 to 4 nitrogen atom(s) which has phenyl having (C₁-C₆)alkoxy, unsaturated 3 to 8-membered heteromonocyclic group containing 1 to 4 nitrogen atom(s) which has phenyl having (C₇-C₁₄)alkoxy(C₁-C₆)alkyl, saturated 3 to 8-membered heteromonocyclic group containing 1 to 4 nitrogen atom(s) which has phenyl having (C₄-C₆)alkyl, unsaturated 3 to 8-membered heteromonocyclic group containing 1 to 4 nitrogen atom(s) which has phenyl having saturated 3 to 8-membered heteromonocyclic group containing 1 to 4 nitrogen atom(s), unsaturated 3 to 8-membered heteromonocyclic group containing 1 to 4 nitrogen atom(s) which has phenyl substituted with (C₁-C₆)alkyl having saturated 3 to 8-membered heteromonocyclic group containing 1 to 4 nitrogen atom(s) having phenyl, unsaturated 3 to 8-membered heteromonocyclic group containing 1 to 4 nitrogen atom(s) which has saturated 3 to 8-membered heteromonocyclic group containing 1 to 4 nitrogen atom(s) having phenyl, phenyl substituted with phenyl which has cyclo(C₄-C₆)alkyloxy, phenyl substituted with phenyl which has (C₁-C₆)alkoxy(C₁-C₆)alkyl, phenyl substituted with phenyl which has (C₁-C₆)alkoxy(C₁-C₆)alkoxy, phenyl substituted with phenyl which has (C₁-C₆)alkoxy(C₁-C₆)alkoxy(C₁-C₆)alkyl, phenyl substituted with phenyl which has (C₁-C₆)alkoxy(C₁-C₆)alkoxy(C₁-C₆)-alkoxy, phenyl substituted with phenyl which has saturated 3 to 8-membered heteromonocyclic group containing 1 to 4 nitrogen atom(s) having cyclo(C₄-C₆)alkyl, phenyl substituted with phenyl which has saturated 3 to 8-membered heteromonocyclic group containing 1 to 4 nitrogen atom(s) substituted with cyclo(C₄-C₆)alkyl having di(C₁-C₄)alkyl, phenyl which has cyclo(C₄-C₆)alkyloxy, phenyl which has (C₁-C₆)alkoxy(C₇-C₁₄)alkylthio, phenyl which has (C₁-C₆)alkoxy having saturated 3 to 8-membered heteromonocyclic group containing 1 to 4 nitrogen atom(s), phenyl which has (C₁-C₆)alkoxy having saturated 3 to 8-membered heteromonocyclic group containing 1 or 2 oxygen atom(s) and 1 to 3 nitrogen atom(s), phenyl which has (C₁-C₆)alkoxy having saturated 3 to 8-membered heteromonocyclic group containing 1 or 2 oxygen atom(s) and 1 to 3 nitrogen atom(s) having di(C₁-C₄)alkyl, phenyl which has (C₁-C₆)alkoxy having saturated 3 to 8-membered heteromonocyclic group containing 1 or 2 sulfur atom(s) and 1 to 3 nitrogen atom(s), cyclo(C₄-C₆)alkyl which has (C₁-C₆)alkyl, cyclo(C₄-C₆)alkyl which has phenyl, indanyl, phenyl substituted with saturated 3 to 8-membered heteromonocyclic group containing 1 or 2 oxgen atom(s) and 1 to 3 nitrogen atom(s), phenyl substituted with saturated 3 to 8-membered heteromonocyclic group containing 1 to 4 nitrogen atom(s) having (C₁-C₆)alkyl, phenyl substituted with saturated 3 to 8-membered heteromonocyclic group containing 1 or 2 oxygen atom(s) and 1 to 3 nitrogen atom(s) having di(C₁-C₄)alkyl, and phenyl substituted with saturated 3 to 8-membered heteromonocyclic group containing 1 or 2 sulfur atom(s) and 1 to 3 nitrogen atom(s),

and the most preferred one may be phenyl having octyloxy, phenyl having hexyloxy, phenyl having cyclohexyl, phenyl having piperidyl, cyclohexyl having cyclohexyl, phenyl having methoxyoctyloxy, phenyl having methoxyheptyloxy, phenyl having butoxy, phenyl having pentyloxy, phenyl substituted with phenyl having methoxy, phenyl substituted with phenyl having propyloxy, phenyl substituted with phenyl having butoxy, phenyl substituted with phenyl having pentyloxy, phenyl substituted with phenyl having hexyloxy, phenyl substituted with phenyl having heptyloxy, phenyl substituted with phenyl which has propyloxy having piperidyl, phenyl having methoxyhexyloxy, isoxazolyl having decyloxy, phenyl substituted with phenyl having phenyloxypropyloxy, phenyl substituted with phenyl having propenyloxy, phenyl substituted with phenyl having phenyloxybutoxy, phenyl substituted with phenyl having methoxyoctyloxy, phenyl substituted with phenyl which has propoxy having dimethyl, phenyl having phenyloxypropoxy, phenyl having phenyloxybutoxy, phenyl having phenyloxypentyloxy, phenyl having methoxypentyloxy, phenyl having methoxyheptyloxy, pyridyl having piperidyl, phenyl having cyclohexyloxy, phenyl which has piperidyl having propoxy, phenyl which has piperidyl having cyclohexyl, phenyl which has piperidyl having phenylmethoxy, phenyl which has piperazinyl having cyclohexyl, phenyl which has piperazinyl substituted with cyclohexyl having dimethyl, phenyl which has piperazinyl substituted with cyclohexyl having methyl, phenyl which has piperidyl substituted with methoxy and chlorophenyl, phenyl which has piperidyl substituted with phenyl, phenyl which has piperazinyl substituted with phenyl, phenyl which has thiadiazolyl substituted with pentyloxyphenyl, pyrazolyl which has hexyloxyphenyl, pyrazolyl which has heptyloxymethylphenyl, piperazinyl which has phenyl having cyclohexyl, pyrazolyl which has phenyl having piperidyl, pyrazolyl which has phenyl having pyrrolidinyl, pyrazolyl which has phenyl substituted with piperazinylmethyl having phenyl, pyridyl which has piperidyl having phenyl, phenyl substituted with phenyl which has cyclohexyloxy, phenyl substituted with phenyl which has ethoxymethyl, phenyl substituted with phenyl which ass ethoxypropoxy, phenyl substituted with phenyl which has ethoxyethoxy, phenyl substituted with phenyl which has methoxypropoxy, phenyl substituted with phenyl which has methoxyethoxy, phenyl substituted with phenyl which has methoxypentyloxy, phenyl substituted with phenyl which has methoxyethoxymethyl, phenyl substituted with phenyl which has methoxyethoxyethoxy, phenyl substituted with phenyl which has piperazinyl having cyclohexyl, phenyl substituted with phenyl which has morpholinyl having dimethyl, phenyl which has cyclohexyloxy, phenyl which has methoxyheptylthio, phenyl which has piperidinobutoxy, phenyl which has piperidinopentyloxy, phenyl which has piperidinohexyloxy, phenyl which has morpholinopentyloxy, phenyl which has morpholinopentyloxy having dimethyl, phenyl which has morpholinohexyloxy having dimethyl, phenyl which has thiomorpholinopentyloxy, cyclohexyl which has pentyl, cyclohexyl which has phenyl, indanyl, phenyl having piperidyl, phenyl having morpholinyl, phenyl having thiomorpholino, phenyl substituted with phenyl having methoxybutoxy, phenyl substituted with piperazinyl having ethyl, and phenyl substituted with morpholinyl having dimethyl.

Suitable example of “aroyl substituted with heterocyclic group which may have one or more suitable substituent(s)” may be benzoyl substituted with piperazinyl which has phenyl having octyloxy, benzoyl substituted with piperazinyl which has phenyl having hexyloxy, benzoyl substituted with thiadiazolyl which has phenyl having hexyloxy, benzoyl substituted with oxadiazolyl which has phenyl having hexyloxy, benzoyl substituted with piperazinyl which has phenyl having cyclohexyl, benzoyl substituted with thiadiazolyl which has phenyl having methoxyoctyloxy, benzoyl substituted with thiadiazolyl which has phenyl having piperidyl, benzoyl substituted with piperazinyl which has cyclohexyl having cyclohexyl, benzoyl substituted with piperazinyl which has phenyl having methoxyoctyloxy, benzoyl substituted with piperazinyl which has phenyl having methoxyheptyloxy, benzoyl substituted with imidazothiadiazolyl which has phenyl having butyloxy, benzoyl substituted with imidazothiadiazolyl which has phenyl having pentyloxy, benzoyl substituted with oxadiazolyl which has phenyl substituted with phenyl having methoxy, benzoyl substituted with oxadiazolyl which has phenyl substituted with phenyl having propyloxy, benzoyl substituted with oxadiazolyl which has phenyl substituted with phenyl having butyloxy, benzoyl substituted with oxadiazolyl which has phenyl substituted with phenyl having pentyloxy, benzoyl substituted with oxadiazolyl which has phenyl substituted with phenyl having hexyloxy, benzoyl substituted with oxadiazolyl which has phenyl substituted with phenyl having heptyloxy, benzoyl substituted with oxadiazolyl which has phenyl substituted with phenyl which has propyloxy having piperidyl, benzoyl substituted with thiadiazolyl which has phenyl having methoxyhexyloxy, benzoyl substituted with oxadiazolyl which has pyrazolyl having decyl, benzoyl substituted with thiadiazolyl which has pyrazolyl having decyl, benzoyl substituted with oxadiazolyl which has phenyl substituted with phenyl having phenyloxypropyloxy, benzoyl substituted with oxadiazolyl which has phenyl substituted with phenyl having propenyloxy, benzoyl substituted with thiadiazolyl which has phenyl having methoxyhexyloxy, benzoyl substituted with oxadiazolyl which has phenyl substituted with phenyl having phenyloxybutyloxy, benzoyl substituted with oxadiazolyl which has phenyl substituted with phenyl having methoxyoctyloxy, benzoyl substituted with oxadiazolyl which has phenyl substituted with phenyl which has propyloxy having dimethylmorpholinyl, benzoyl substituted with thiadiazolyl which has phenyl having phenyloxybutyloxy, benzoyl substituted with thiadiazolyl which has phenyl having phenyloxypentyloxy, benzoyl substituted with thiadiazolyl which has phenyl having phenyloxypropyloxy, benzoyl substituted with thiadiazolyl which has phenyl having methoxypentyloxy, benzoyl substituted with thiadiazolyl which has phenyl having methoxyheptyloxy, benzoyl substituted with thiadiazolyl which has pyridyl having piperidyl, benzoyl substituted with imidazothiadiazolyl which has phenyl having pentyloxy, benzoyl substituted with imidazothiadiazolyl which has phenyl having cyclohexyloxy, benzoyl substituted with isoxazolyl which has phenyl having pentyloxy, benzoyl substituted with thiadiazolyl having phenyl which has piperidyl having propoxy, benzoyl substituted with thiadiazolyl having phenyl which has piperidyl having cyclohexyloxy, benzoyl substituted with thiadiazolyl having phenyl which has piperidyl having phenylmethoxy, benzoyl substituted with imidazothiadiazolyl having phenyl which has piperazinyl having cyclohexyl, benzoyl substituted with thiadiazolyl having phenyl which has piperazinyl substituted with cyclohexyl having dimethyl, benzoyl substituted with thiadiazolyl having phenyl which has piperazinyl having cyclohexyl, benzoyl substituted with thiadiazolyl having phenyl which has piperazinyl substituted with cyclohexyl having methyl, benzoyl substituted with thiadiazolyl having phenyl which has piperidyl substituted with methoxy and chlorophenyl, benzoyl substituted with thiadiazolyl having phenyl which has piperidyl substituted with phenyl, benzoyl substituted with thiadiazolyl having phenyl which has piperazinyl substituted with phenyl, benzoyl substituted with thiadiazolyl having phenyl which has thiadiazolyl substituted with pentyloxyphenyl, benzoyl substituted with thiadiazolyl having pyrazolyl which has hexyloxyphenyl, benzoyl substituted with thiadiazolyl having pyrazolyl which has heptyloxymethylphenyl, benzoyl substituted with piperidyl having piperazinyl which has phenyl having cyclohexyl, benzoyl substituted with thiadiazolyl having pyrazolyl which has phenyl having piperidyl, benzoyl substituted with thiadiazolyl having pyrazolyl which has phenyl having pyrrolidinyl, benzoyl substituted with thiadiazolyl having pyrazolyl which has phenyl substituted with piperazinylmethyl having phenyl, benzoyl substituted with thiadiazolyl having pyridyl which has piperidyl having phenyl, benzoyl substituted with thiadiazolyl having phenyl substituted with phenyl which has cyclohexyloxy, benzoyl substituted with thiadiazolyl having phenyl substituted with phenyl which has ethoxymethyl, benzoyl substituted with thiadiazolyl having phenyl substituted with phenyl which has ethoxypropoxy, benzoyl substituted with thiadiazolyl having phenyl substituted with phenyl which has ethoxyethoxy, benzoyl substituted with thiadiazolyl having phenyl substituted with phenyl which has methoxypropoxy, benzoyl substituted with thiadiazolyl having phenyl substituted with phenyl which has methoxyethoxy, benzoyl substituted with piperazinyl having phenyl substituted with phenyl which has methoxypentyloxy, benzoyl substituted with thiadiazolyl having phenyl substituted with phenyl which has methoxyethoxymethyl, benzoyl substituted with thiadiazolyl having phenyl substituted with phenyl which has methoxyethoxyethoxy, benzoyl substituted with thiadiazolyl having phenyl substituted with phenyl which has piperazinyl having cyclohexyl, benzoyl substituted with thiadiazolyl having phenyl substituted with phenyl which has morpholinyl having dimethyl, benzoyl substituted with oxadiazolyl which has phenyl having cyclohexyloxy, benzoyl substituted with thiadiazolyl which has phenyl having cyclohexyloxy, benzoyl substituted with piperazinyl which has phenyl having cyclohexyloxy, benzoyl substituted with piperazinyl which has phenyl having methoxyheptylthio, benzoyl substituted with imidazothiadiazolyl which has phenyl having piperidinobutoxy, benzoyl substituted with imidazothiadiazolyl which has phenyl having piperidinopentyloxy, benzoyl substituted with imidazothiadiazolyl which has phenyl having piperidinohexyloxy, benzoyl substituted with imidazothiadiazolyl which has phenyl having morpholinopentyloxy, benzoyl substituted with imidazothiadiazolyl having phenyl which has morpholinopentyloxy having dimethyl, benzoyl substituted with imidazothiadiazolyl having phenyl which has morpholinohexyloxy having dimethyl, benzoyl substituted with imidazothiadiazolyl having phenyl which has thiomorpholinopentyloxy, benzoyl substituted with piperazinyl which has cyclohexyl having pentyl, benzoyl substituted with piperazinyl which has cyclohexyl having phenyl, benzoyl substituted with piperazinyl which has indanyl, benzoyl substituted with imidazothiadiazolyl having phenyl which has piperazinyl having ethyl, benzoyl substituted with imidazothiadiazolyl which has phenyl having butoxy, benzoyl substituted with imidazothiadiazolyl which has phenyl having methoxypentyloxy, benzoyl substituted with piperazinyl which has phenyl having cyclohexyl, dimethylbenzoyl substituted with thiadiazolyl which has phenyl having methoxyhexyloxy, naphthoyl substituted with oxadiazolyl having phenyl substituted with phenyl having butoxy, naphthoyl substituted with thiadiazolyl which has phenyl having methoxyhexyloxy, benzoyl substituted with thiazolyl which has phenyl having pentyloxy, benzoyl substituted with thiazolyl which has phenyl having hexyloxy, benzoyl substituted with thiazolyl which has phenyl having heptyloxy, benzoyl substituted with thiazolyl having phenyl substituted with phenyl having propoxy, benzoyl substituted with imidazothiadiazolyl which has phenyl having methoxyhexyloxy, benzoyl substituted with imidazothiadiazolyl which has phenyl having methoxyheptyloxy, benzoyl substituted with imidazothiadiazolyl which has phenyl having methoxyoctyloxy, benzoyl substituted with imidazothiadiazolyl which has phenyl having morpholino, benzoyl substituted with imidazothiadiazolyl which has phenyl having dimethylmorpholino, benzoyl sustituted with imidazothiadiazolyl which has phenyl having thiomorpholino, benzoyl substituted with imidazothiadiazolyl which has phenyl having pentyloxy, benzoyl substituted with imidazothiadiazolyl which has phenyl having hexyloxy, benzoyl substituted with thiadiazolyl which has phenyl having cyclohexyl, benzoyl substituted with oxadiazolyl which has phenyl having cyclohexyl, benzoyl substituted with thiadiazolyl which has phenyl substituted with phenyl having propoxy, benzoyl substituted with thiadiazolyl which has phenyl substituted with phenyl having ethoxy, benzoyl substituted with thiadiazolyl which has phenyl substituted with phenyl having methoxybutoxy, and benzoyl substituted with thiadiazolyl which has phenyl substituted with phenyl having butoxy.

The process for preparing the object compound (I) or a salt thereof of the present invention is explained in detail in the following.

### Process 1

The compound (Ia) or a salt thereof of the present invention can be produced by fermentation of the compound (Ia) or a salt thereof-producing strain belonging to the genus Coleophoma such as Coleophoma sp. F-11899 in a nutrient medium.

### (i) Microorganism

Particulars of the microorganism used for producing the compound (Ia) or a salt thereof is explained in the following.

The strain F-11899 was originally isolated from a solid sample collected at Iwaki-shi, Fukushima-ken, Japan. This organism grew rather restrictedly on various culture media, and formed dark gray to brownish Grey colonies. Anamorph (conidiomata) produced on a steam-sterilized leaf segment affixed on a Miura's LCA plate¹) or a corn meal agar plate by inoculating the isolate, while neither teleomorph nor anamorph formed on the agar media. Its morphological, cultural and physiological characteristics are as follows.

Cultural characteristics on various agar media are summarized in Table 1. Cultures on potato dextrose agar grew rather rapidly, attaining 3.5-4.0 cm in diameter after two weeks at 25° C. This colony surface was plane, felty, somewhat wrinkly and brownish gray. The colony center was pale gray to brownish gray, and covered with aerial hyphae. The reverse color was dark gray. Colonies on malt extract agar grew more restrictedly, attaining 2.5-3.0 cm in diameter under the same conditions. The surface was plane, thin to felty and olive brown. The colony center was yellowish gray, and covered with aerial hyphae. The reverse was brownish gray.

The morphological characteristics were determined on basis of the cultures on a sterilized leaf affixed to a Miura's LCA plate. Conidiomata formed on the leaf segment alone. They were pycnidial, superficial, separate, discoid to ampulliform, flattened at the base, unilocular, thin-walled, black, 90-160(−200) μm in diameter and 40-70 μm high. Ostiole was often single, circular, central, papillate, 10-30 μm in diameter and 10-20 μm high. Conidiophores formed from the lower layer of inner pycnidial walls. They were hyaline, simple or sparingly branched, septate and smooth. Conidiogenous cells were enteroblastic, phialidic, determinate, ampulliform to obpyriform, hyaline, smooth, 5-8×4-6 μm, with a collarette. The collarettes were campanulate to cylindrical, and 14-18×3-5 μm. Conidia were hyaline, cylindrical, thin-walled, aseptate, smooth and 14-16(−18)×2-3 μm.

The vegetative hyphae were septate, brown, smooth and brnached. The hyphal cells were cylindrical and 2-7 μm thick. The chlamydospores were absent.

The strain F-11899 had a temperature range for growth of 0° C. to 31° C. and an optimum temperature of 23° C. to 27° C. on potato dextrose agar.

The above characteristics indicate that the strain F-11899 belongs to the order Coelomycetes^{2), 3), 4)}. Thus, we named the strain “Coelomycetes strain F-11899”.

TABLE 1
Cultural characteristics of the Strain F-11899

| Medium | Cultural characteristics |
|---|---|
| Malt extract agar | G: Rather restrictedly, 2.5-3.0 cm |
| (Blakeslee 1915) | S: Circular, plane, thin to felty, |
| | olive brown (4F5), arising aerial |
| | hyphae at the center (yellowish gray |
| | (4B2)) |
| | R: Brownish gray (4F2) |
| Potato dextrose agar | G: Rather rapidly, 3.5-4.0 cm |
| (Difco 0013) | S: Circular, plane, felty, somewhat |
| | wrinkly, brownish gray (4F2), |
| | arising aerial hyphae at the center |
| | (pale gray (4B1) to brownish gray |
| | (4F2)) |
| | R: Dark gray (4F1) |
| Czapeck's solution | G: Very restrictedly, 1.0-1.5 cm |
| Agar (Raper and | S: Irregular, thin, scanty, immersed, |
| Thom 1949) | subhyaline to white |
| | R: Subhyaline to white |
| Sabouraud dextrose | G: Restrictedly, 2.0-2.5 cm |
| Agar (Difco 0109) | S: Circular, plane, thin, white, |
| | sectoring, light brown (6D5) at the |
| | colony center |
| | R: Pale yellow (4A3) |
| Oatmeal agar | G: Fairly rapidly, 4.0-4.5 cm |
| (Difco 0552) | S: Circular, plane, felty to cottony, |
| | dark gray (4F1) to brownish gray |
| | (4F2) |
| | R: Brownish gray (4D2) |
| Emerson Yp Ss agar | G: Restrictedly, 2.0-2.5 cm |
| (Difco 0739) | S: Circular to irregular, plane, felty, |
| | dark gray (4F1) to brownish gray |
| | (4F2) |
| | R: Medium gray (4E1) to dark gray (4F1) |
| Corn meal agar | G: Rather restrictedly, 2.5-3.0 cm |
| (Difco 0386) | S: Circular, plane, thin to felty, dark |
| | gray (2F1) to olive (2F3) |
| | R: Dark gray (2F1) to olive (2F3) |
| MY20 agar | G: Restrictedly, 1.5-2.0 cm |
| | S: Circular to irregular, thin, |
| | sectoring, yellowish white (4A2) |
| | R: Pale yellow (4A3) to orange white |
| | (5A2) |

Abbreviations:
G: growth, measuring colony size in diameter
S: colony surface
R: reverse

These characteristics were observed after 14 days of incubation at 25° C. The color descriptions were based on the Methuen Handbook of Color⁵⁾.

1) Miura, K. and M. Y. Kudo: An agar-medium for aquatic Hyphomycetes., Trans. Ycolo. Soc. Japan, 11:116-118, 1970.

2) Arx, J. A. von: The Genera of Fungi—Sporulating in Pure Culture (3rd ed.), 315 p., J. Cramer, Vaduz, 1974.

3) Sutton, B. C.: The Coelomycetes—Fungi Imperfecti with Pycnidia, Acervuli and Stromata., 696 p., Commonwealth Mycological Institute, Kew, 1980.

4) Hawksworth, D. L., B. C. Sutton and G. C. Ainsworth:

Dictionary of the Fungi (7th ed.), 445 p., Commonwealth Mycological Institute, Kew., 1983.

5) Kornerup, A. and Wanscher, J. H.: Methuen Handbook of Color (3rd ed.), 252 p., Methuen, London, 1983.

A culture of Coelomycetes strain F-11899 thus named has been deposited with International Patent Organism Depositary (former name: Fermentation Research Institute Agency of Industrial Science and Technology), (1-1, Higashi 1-chome, Tsukuba-shi IBARAKI 305-8566 JAPAN), on Oct. 26, 1989 under the number of FERM BP-2635.

After that, however, we have further studied the classification of the strain F-11899, and have found that the strain F-11899 resembled Coleophoma empetri (Rostrup) Petrak 1929^{2), 3), 4)} belonging to the order Coelomycetes, but differed in some pycnidial characteristics: globose or flattened at the base, immersed, and not papillate.

Considering these characteristics, we classified this strain in more detail and renamed it as “Coleophoma sp. F-11899”.

In this connection, we have already taken step to amend the name, “Coelomycetes strain F-11899” to “Coleophoma sp. F-11899” with International Patent Organism Depsitory (former name: Fermentation Research Institure Agency of Science and Technology), on Sep. 21, 1990.

### (ii) Production of the Compound (Ia) or a Salt thereof

The compound (Ia) or a salt thereof of the present invention is produced when the compound (Ia) or a salt thereof-producing strain belonging to the genus Coleophoma is grown in a nutrient medium containing sources of assimilable carbon and nitrogen under aerobic conditions (e.g. shaking culture, submerged culture, etc.). The preferred sources of carbon in the nutrient medium are carbohydrates such as glucose, sucrose, starch, fructose or glycerin, or the like.

The preferred sources of nitrogen are yeast extract, peptone, gluten meal, cotton seed flour, soybean meal, corn steep liquor, dried yeast, wheat germ, etc., as well as inorganic and organic nitrogen compounds such as ammonium salts (e.g. ammonium nitrate, ammonium sulfate, ammonium phosphate, etc.), urea or amino acid, or the like.

The carbon and nitrogen sources, though advantageously employed in combination, need not to be used in their pure form becouse less pure materials, which contain traces of growth factors and considerable quantities of mineral nutrients, are also suitable for use.

When desired, there may be added to the medium mineral salts such as sodium or calcium carbonate, sodium or potassium phosphate, sodium or potassium chloride, sodium or potassium iodide, magnesium salts, copper salts, zinc salts, or cobalt salts, or the like. If necessary, especially when the culture medium foams seriously a defoaming agent, such as liquid paraffin, fatty oil, plant oil, mineral oil or silicone, or the like may be added.

As in the case of the preferred methods used for the production of other biologically active substances in massive amounts, submerged aerobic cultural conditions are preferred for the production of the compound (Ia) or a salt thereof in massive amounts.

For the production in small amounts, a shaking or surface culture in a flask or bottle is employed.

Further, when the growth is carried out in large tanks, it is preferable to use the vegetative form of the organism for inoculation in the production tanks in order to avoid growth lag in the process of production of the compound (Ia) or a salt thereof. Accordingly, it is desirable first to produce a vegetative inoculum of the organism by inoculating a relatively small quantity of culture medium with spores or mycelia of the organism and culturing said inoculated medium, and then to transfer the cultured vegetative inoculum to large tanks. The medium, in which the vegetative inoculum is produced, is substantially the same as or different from the medium utilized for the production of the compound (Ia) or a salt thereof.

Agitation and aeration of the culture mixture may be accomplished in a variety of ways. Agitation may be provided by a propeller or similar mechanical agitation equipment, by revolving or shaking the fermentor, by various pumping equipment or by the passage of sterile air through the medium. Aeration may be effected by passing sterile air through the fermentation mixture.

The fermentation is usually conducted at a temperature between about 10° C. and 40° C., preferably 20° C. to 30° C., for a period of about 50 hours to 150 hours, which may be varied according to fermentation conditions and scales.

When the fermentation is completed, the culture broth is then subjected for recovery of the compound (Ia) or a salt thereof to various procedures conventionally used for recovery and purification of biological active substances, for instance, solvent extraction with an appropriate solvent or a mixture of some solvents, chromatography on recrystallization from an appropriate solvent or a mixture of some solvents, or the like.

According to this invention, in general, the compound (Ia) or a salt thereof is found both in the cultured mycelia and cultured broth. Accordingly, then the compound (Ia) or a salt thereof is removed from the whole broth by means of extraction using an appropriate organic solvent such as acetone or ethyl acetate, or a mixture of these solvents, or the like.

The extract is treated by a conventional manner to provide the compound (Ia) or a salt thereof, for example, the extract is concentrated by evaporation or distillation to a smaller amount and the resulting residue containing active material, i.e. the compound (Ia) or a salt thereof is purified by conventional purification procedures, for example, chromatography on recrystallization from an appropriate solvent or a mixture of some solvents.

When the object compound is isolated as a salt of the compound (Ia), it can be converted to the free compound (Ia) or another salt of the compound (Ia) according to a conventional manner.

### Process 2

The compound (Ib) or a salt thereof can be prepared by reacting the compound (Ia) or a salt thereof.

The reaction can be carried out in a conventional manner, namely, chemical reduction or catalytic reduction.

Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, hydride transfer reagent such as aluminum hydride compound (e.g. lithium aluminum hydride, lithium hydridotri-t-butoxyaluminate, etc.), borohydride compound (e.g. sodium borohydride, sodium cyanoborohydride, etc.) or the like.

Suitable catalysts to be used in catalytic reduction are conventional ones such as platinum catalyst [e.g. platinum plate, spongy platinum, platinum black, colloidal platinum, platinum oxide, platinum wire, etc.], palladium catalyst [e.g. spongy palladium, palladium black, palladium oxide, palladium on carbon, colloidal palladium, palladium on barium sulfate, palladium on barium carbonate, etc.], nickel catalyst [e.g. reduced nickel, nickel oxide, Raney nickel, etc.], cobalt catalyst [e.g. reduced cobalt, Raney cobalt, etc.], iron catalyst [e.g. reduced iron, Raney iron, etc.], copper catalyst [e.g. reduced copper, Raney copper, Ullman copper, etc.] or the like.

The reaction of this process is usually carried out in a solvent such as water, alcohol [e.g. methanol, ethanol, propanol, etc.], acetic acid, diethyl ether, dioxane, tetrahydrofuran, methylene chloride, etc. or a mixture thereof.

The reaction is preferably carried out under somewhat milder conditions such as under cooling to warming.

### Process 3

The compound (Id) or a salt thereof can be prepared by reacting a compound (Ic) or a salt thereof to elimination reaction of N-acyl group.

This reaction is carried out in accordance with a conventional method such as hydrolysis, reduction, reaction with an enzyme or the like.

The hydrolysis is preferably carried out in the presence of a base or an acid including Lewis acid. Suitable base may include an inorganic base and an organic base such as an alkali metal [e.g. sodium, potassium, etc.], an alkaline earth metal [e.g. magnesium, calcium, etc.], the hydroxide or carbonate or bicarbonate thereof, trialkylamine [e.g. trimethylamine, triethylamine, etc..], picoline, 1,5-diazabicyclo[4.3.0]non-5-ene, 1,4-diazabicyclo[2.2.2]octane, 1,8-diazabicyclo[5.4.0]undec-7-ene, or the like.

Suitable acid may include an organic acid [e.g. formic acid, acetic acid, propionic acid, trichloroacetic acid, trifluoroacetic acid, etc.] and an inorganic acid [e.g. hydrochloric acid, hydrobromic acid, sulfuric acid, hydrogen chloride, hydrogen bromide, etc.]. The elimination using Lewis acid such as trihaloacetic acid [e.g. trichloroacetic acid, trifluoroacetic acid, etc.], or the like, is preferably carried out in the presence of cation trapping agents [e.g. anisole, phenol, etc.].

The reaction is usually carried out in a solvent such as water, an alcohol [e.g. methanol, ethanol, etc.], methylene chloride, tetrahydrofuran, a mixture thereof or any other solvent which does not adversely influence the reaction. A liquid base or acid can be also used as the solvent. The reaction temperature is not critical and the reaction is usually carried out under cooling to warming.

The reduction method applicable for the elimination reaction may include chemical reduction and catalytic reduction.

Suitable reducting agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium sulfate, palladium on barium carbonate, etc.], nickel catalysts [e.g. reduced nickel, nickel oxide, Raney nickel, etc.], cobalt catalysts [e.g. reduced cobalt, Raney cobalt, etc.], iron catalysts [e.g. reduced iron, Raney iron, etc.], copper catalysts [e.g. reduced copper, Raney copper, Ullman copper, etc.] and the like. The reduction is usually carried out in a conventional solvent which does not adversely influence the reaction such as water, methanol, ethanol, propanol, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical, and the reaction is usually carried out under cooling to warming.

The reaction with an enzyme can be carried out by reacting the compound (Ic) or a salt thereof with an enzyme suitable for the elimination reaction of N-acyl group.

Suitable example of said enzyme may include the one produced by certain microorganisms of the Streptomycetaceae, the Actinoplanaceae, the Oidiodendron or the Verticillium, for example, Streptomyces sp. No.6907 (FERM BP-5809), Streptomyces anulatus No.4811 (FERM BP-5808), Streptomyces anulatus No.8703 (FERM BP-5810), Actinoplanes utahensis IFO-13244, Actinoplanes utahensis ATCC 12301, Actinoplanes missenrieneses NRRL 12053, Oidiodendron sp. No.30084 (FERM BP-5943), Verticillium sp. No.30085 (FERM BP-5944), or the like; and the like.

This elimination reaction is usually carried out in a solvent such as phosphate buffer, Tris-HCl buffer or any other solvent which does not adversely influence the reaction.

The reaction temperature is not critical and the reaction can be carried out at room temperature or under warming.

### Process 4

The compound (Ie) or a salt thereof can be prepared by reacting the compound (Id) or its reactive derivative at the amino group or a salt thereof with the compound (II) or its reactive derivative at the carboxy group or a salt thereof.

Suitable reactive derivative at the carboxy group of the compound (II) may include an acid halide, an acid anhydride, an activated amide, an activated ester, and the like. Suitable examples of the reactive derivatives may be an acid chloride; an acid azide; a mixed acid anhydride with an acid such as substituted phosphoric acid [e.g., dialkylphosphoric acid, phenylphosphoric acid, diphenylphosphoric acid, dibenzylphosphoric acid, halogenated phosphoric acid, etc.], dialkylphosphorous acid, sulfurous acid, thiosulfuric acid, sulfuric acid, sulfonic acid [e.g., methanesulfonic acid, etc.], aliphatic carboxylic acid [e.g., acetic acid, propionic acid, butyric acid, isobutyric acid, pivaric acid, pentanoic acid, isopentanoic acid, 2-ethylbutyric acid, trichloroacetic acid, etc.]; or aromatic carboxylic acid [e.g., benzoic acid, etc.]; a symmetrical acid anhydride; an activated amide with imidazole, 4-substituted imidazole, dimethylpyrazole, triazole, tetrazole or 1-hydroxy-1H-benzotriazole; or an activated ester [e.g., cyanomethyl ester, methoxymethyl ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenyl thioester, p-nitrophenyl thioester, p-cresyl thioester, carboxmethyl thioester, pyranyl ester, pyridyl ester, piperidyl ester, 8-quinolyl ethioester, etc.], or an ester with a N-hydroxy compound [e.g. N,N-dimethylhydroxylamine, 1-hydroxy-2-(1H)-pyridone, N-hydroxysuccinimide, N-hydroxyphthalimide, 1-hydroxy-1H-benzotriazole, etc.], and the like. These reactive derivatives can optionally be selected from them according to the mind of the compound (II) to be used.

Suitable salts of the compound (II) and its reactive derivative can be referred to the ones as exemplified for the compound (I).

The reaction is usually carried out in a conventional solvent such as water, alcohol [e.g., methanol, ethanol, etc.], acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction. These conventional solvent may also be used in a mixture with water.

In this reaction, when the compound (II) is used in a free acid form or its salt form, the reaction is preferably carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)carbodiimide; N,N′-diethylcarbodiimide, N,N′-diisopropylcarbodiimide; N-ethyl-N′-(3-dimethylaminopropyl)carbodiimide, N,N-carbonylbis-(2-methylimidazole); pentamethyleneketene-N-cyclohexylimine; diphenylketene-N-cyclohexylimine; ethoxyacetylene; 1-alkoxy-2-chloroethylene; trialkyl phosphite; ethyl polyphosphate; isopropyl polyphosphate; phosphorus oxychloride (phosphoryl chloride); phosphorus trichloride; thionyl chloride; oxalyl chloride; lower alkyl haloformate [e.g., ethyl chloroformate, isopropyl chloroformate, etc.]; triphenylphosphine; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, trichloromethyl chloroformate, phosphorous oxychloride, methanesulfonyl chloride, etc.; or the like.

The reaction may also be carried out in the presence of an inorganic or organic base such as an alkali metal carbonate, alkali metal bicarbonate, di(lower)alkylamine (e.g., diisopropylethylamine, etc.), tri(lower)alkylamine (e.g., triethylamine, etc.), pyridine, di(lower)alkylaminopyridine (e.g., 4-dimethylaminopyridine, etc.), N-(lower)alkylmorpholine, N,N-di(lower)alkylbenzylamine, or the like.

The reaction temperature is not critical, and the reaction is usually carried out under cooling to warming.

The compounds obtained by the above Processes 1 to 4 can be isolated and purified by a conventional method such as pulverization, recrystallization, column-chromatography, high-performance liquid chromatography (HPLC), reprecipitation, or the like.

The compounds obtained by the above Processes 1 to 4 may be obtained as solvated compound (e.g., hydrate, ethanolate, etc.), and such as solvated compound is included within the scope of the present invention.

It is to be noted that each of the compounds obtained by the above Processes 1 to 4 may include one or more stereoisomer(s) such as optical isomer(s) and geometrical isomer(s) due to asymmetric carbon atom(s) and double bond(s), and all such isomer(s) and the mixture thereof are included within the scope of the present invention.

The compounds obtained by the above Processes 1 to 4 may include both its crystal form and non-crystal form.

It should be understood that the compounds obtained by the above Processes 1 to 4 may include the prodrug form.

The compounds obtained by the above Processes 1 to 4 may be used in combination with the known antifungal agents such as the azoles (e.g. fluconazole, itraconazole, etc.) or polyenes (e.g. amphotericin B, etc.).

The present patent, patent applications and publications cited herein are incorporated by reference.

### Biological Property of the Compound (I) of the Present Invention

In order to show the usefulness of the polypeptide compound (I) of the present invention, the biological data of the representative compound is explained in the following.

### Test (Antimicrobial Activity)

### Test Method

The antifungal susceptibility assays were performed by the microdilution method according to M27-A guidelines recommended by the National Committee for Clinical Laboratory Standards (NCCLS) to determine the MICs of the compounds.

RPMI 1640 medium with L-glutamine, without sodium bicarbonate and buffered with 165 mM morpholinepropanesulfonic acid buffer (pH 7.0) was used as a test medium. Inoculum suspension of 10⁶ CFU/ml were prepared by a hemocytometric procedure and diluted to obtain an inoculum size of approximately 0.5×10³ to 2.5×10³ CFU/ml. Microplates were incubated at 35° C., and readings were taken when good growth in the growth control. The MICs were defined as the lowest concentrations at which no visible growth was observed.

### Test Result

TABLE 1
[MIC (μg/ml)]

| Test Compound | Object | Object | Object |
|---|---|---|---|
| Microorganism | Compound (8)* | Compound (9)* | Compound (10)* |
| Candida albicans | <0.1 | <0.02 | <0.1 |
| FP 633 | | | |

*Each prepared in Example 8, 9 and 10 mentioned below.

From the test result, it is realized that-the compound (I) of the present invention has an antimicrobial activity (especially, antifungal activity).

In more details, the polypeptide compound (I) of the present invention have an antifungal activity, particularly against the following fungi.

Acremonium;

Absidia (e.g., Absidia corymbifera, etc);

Aspergillus (e.g., Aspergillus clavatus, Aspergillus flavus, Aspergillus fumigatus, Aspergillus nidulans, Aspergillus niger, Aspergillus terreus, Aspergillus versicolor, etc);

Blastomyces (e.g., Blastomyces dermatitidis, etc);

Candida (e.g., Candida albicans, Candida glabrata, Candida guilliermondii, Candida kefyr, Candida krusei, Candida parapsilosis, Candida stellatoidea, Candida tropicalis, Candida utilis, etc.);

Cladosporium (e.g., Cladosporium trichloides, etc);

Coccidioides (e.g., Coccidioides immitis, etc);

Cryptococcus (e.g., Cryptococcus neoformans, etc);

Cunninghamella (e.g., Cunninghamella elegans, etc);

Dermatophyte;

Exophiala (e.g., Exophiala dermatitidis, Exophiala spinifera, etc);

Epidermophyton (e.g., Epidermophyton floccosum, etc);

Fonsecaea (e.g., Fonsecaea pedrosoi, etc);

Fusarium (e.g., Fusarium solani, etc);

Geotrichum (e.g., Geotrichum candiddum, etc);

Histoplasma (e.g., Histoplasma capsulatum var. capsulatum, etc.).

Malassezia (e.g., Malassezia furfur, etc);

Microsporum (e.g., Microsporum canis, Microsporum gypseum, etc);

Mucor;

Paracoccidioides (e.g., Paracoccidioides brasiliensis, etc);

Penicillium (e.g., Penicillium marneffei, etc);

Phialophora;

Pneumocystis (e.g., Pneumocystis carinii, etc);

Pseudallescheria (e.g., Pseudallescheria boydii, etc);

Rhizopus (e.g., Rhizopus microsporus var. rhizopodiformis, Rhizopus oryzae, etc);

Saccharomyces (e.g., Saccharomyces cerevisiae, etc);

Scopulariopsis;

Sporothrix (e.g., Sporothrix schenckii, etc);

Trichophyton (e.g., Trichophyton mentagrophytes, Trichophyton rubrum, etc);

Trichosporon (e.g., Trichosporon asahii, Trichosporon cutaneum, etc).

The above fungi are well-known to cause various infection diseases in skin, eye, hair, nail, oral mucosa, gastrointestinal tract, bronchus, lung, endocardium, brain, meninges, urinary organ, vaginal protion, oral cavity, ophthalmus, systemic, kidney, bronchus, heart, external auditory canal, bone, nasal cavity, paranasal cavity, spleen, liver, hypodermal tissue, lymph doct, gastrointestine, articulation, muscle, tendon, interstitial plasma cell in lung, blood, and so on.

Therefore, the polypeptide compound (I) of the present invention are useful for prevention and treating various infectious diseases, such as dermatophytosis (e.g., trichophytosis, etc), pityriasis versicolor, candidiasis, cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, fungemia, and so on.

The combination use of azoles such as fluconazole, voriconazole, itraconazole, ketoconazole, miconazole, ER 30346 and SCH 56592; polyenes such as amphotericin B, nystatin, liposamal and lipid forms thereof such as Abelcet, AmBisome, and Amphocil; purine or pyrimidine nucleotide inhibitors such as flucytosine; or polyxins such as nikkomycines, in particular nikkomycine Z or nikkomycine X; other chitin inhibitors; elongation factor inhibitors such as sordarin and analogs thereof; mannan inhibitos such as predamycin, bactericidal/permeability-inducing (BPI) protein products such as XMP.97 or XMP.127; or complex carbohydrate antifungal agents such as CAN-296; or the combination use of immunosuppressant such as tacrolimus with the polypeptide compound (I) or a salt thereof is effective against above infectious diseases.

The pharamaceutical composition of the present invention can be used in the form of a pharmaceutical preparation, for example, in solid, semisolid or liquid form, which contains the polypeptide compound (I) or a pharmaceutically acceptable salt thereof, as an active ingredient in admixture with an organic or inorganic carrier or excipient which is suitable for rectal; pulmonary (nasal or buccal inhalation); ocular; external (topical); oral administration; parenteral (including subcutaneous, intravenous and intramuscular) administrations; insufflation (including aerosols from metered dose inhalator); nebulizer; or dry powder inhalator.

The active ingredient may be compounded, for example, with the usual non-toxic, pharmaceutically acceptable carriers in a solid form such as granules, tablets, dragees, pellets, troches, capsules, or suppositories; creams; ointments; aerosols; powders for insufflation; in a liquid form such as solutions, emulsions, or suspensions for injection; ingestion; eye drops; and any other form suitable for use. And, if necessary, there may be included in the above preparation auxiliary substance such as stabilizing, thickening, wetting, emulsifying and coloring agents; perfumes or buffer; or any other commonly may be used as additives.

The polypeptide compound (I) or a pharmaceutically acceptable salt thereof is/are included in the pharmaceutical composition in an amount sufficient to produce the desired antimicrobial effect upon the process or condition of diseases.

For applying the composition to humans, it is preferable to apply it by intravenous, intramuscular, pulmonary, oral administration, eye drop administration or insufflation. While the dosage of therapeutically effective amount of the polypeptide compound (I) varies from and also depends upon the age and condition of each individual patient to be treated, in the case of intravenous administration, a daily dose of 0.01-400 mg of the polypeptide compound (I) per kg weight of human being in the case of intramuscular administration, a daily dose of 0.1-20 mg of the polypeptide compound (I) per kg weight of human being, in case of oral administration, a daily dose of 0.5-50 mg of the polypeptide compound (I) per kg weight of human being is generally given for treating or preventing infectious diseases.

Especially in case of the treatment of prevention of Pneumocystis carinii infection, the followings are to be noted.

For administration by inhalation, the compounds of the present invention are conveniently delivered in the form of an aerosol spray presentation form pressurized as powders which may be formulated and the powder compositions may be inhaled with the aid of an insufflation powder inhaler device. The preferred delivery system for inhalation is a metered dose inhalation aerosol, which may be formulated as a suspension or solution of compound in suitable propellants such as fluorocarbons or hydrocarbons.

Because of desirability to directly treat lung and bronchi, aerosol administration is a preferred method of administration. Insufflation is also a desirable method, especially where infection may have spread to ears and other body cavities.

Alternatively, parenteral administration may be employed using drip intravenous administration.

For administration by intravenous administration, the preferred pharmaceutical composition is the lyophilized form containing the polypeptide compound (I) or its pharmaceutically acceptable salt.

The amount of the polypeptide compound (I) or its pharmaceutically acceptable salt contained in the composition for a single unit dosage of the present invention is 0.1 to 400 mg, more preferably 1 to 200 mg, still more preferably 5 to 100 mg, specifically 5, 10, 15, 20, 25, 30, 35, 40, 45, 50, 55, 60, 65, 70, 75, 80, 85, 90, 95 and 100 mg.

The present invention further provides the following ones.

An article of manufacture, comprising packaging material and the compound (I) identified in the above contained within said packaging material, wherein said the compound (I) is therapeutially effective for preventing or treating infectious diseases caused by pathogenic microorganism, and wherein said packaging material comprises a label or a written material which indicates that said compound (I) can or should be use for preventing or treating infectious diseases caused by pathogenic microorganism.

A commercial package comprising the pharmaceutical composition containing the compound (I) identified in the above and a written matter associated therewith, wherein the written matter states that the compound (I) can or should be used for preventing or treating infectious diseases caused by pathogenic microorganism.

The following Preparations and Examples are given for the purpose of illustrating the present invention in more detail.

### Preparation 1

### Fermentation of Coleophoma sp. F-11899

An aqueous seed medium (160 ml) containing gulanulated sugar 4%, Pharmamedia (TM: cotton seed flour, Traders Protein) 2%, soybean powder 2%, KH₂PO₄ 1.6% and CaCO₃ 0.2% was poured into a 500-ml Erlenmeyer flask and sterilized at 120° C. for 30 minutes.

A loopful of Coleophoma sp. F-11899 was inoculated from a slant culture into the flask. The flask was shaken on a rotary shaker (260 rpm, 5.1 cm-throw) at 25° C. for 6 days. The resultant seed culture was inoculated to 20 liters of sterile production medium consisting of starch hydrolysates Max1000™ 6%, rice-bran oil 3%, soybean powder 2%, wheat germ meal 1% KH₂PO₄ 0.5%, MgSO₄ 0.1%, Adecanol LG-109 (deforming agent, Asahi Denka Co., Ltd.) 0.1% and Silicone KM-70 (deforming agent, Shin-tsu Chemical Co., Ltd.) 0.1% in a 30-liter jar-fermenter. Fermentation was carried out at 25° C. for 13 days under aeration of 20 liters/minute and agitation of 300 rpm.

### Preparation 2

### Production of the acylase of the Streptomyces sp. No.6907

A aqueous seed medium (160 ml) containing modified starch MS#3600™ 6%, soybean meal 3% and CaCO₃ 0.5% was poured into a 500-ml Erlenmeyer flask and sterilized at 120° C. for 30 minutes.

A loopful of Streptomyces sp. No.6907 was inoculated from a slant culture into the flask. The flask was cultured on a rotary shaker (260 rpm, 5.1 cm throw) at 30° C. for 3 days. The resultant seed culture was inoculated to 20 liters of sterile production medium consisting of modified starch MS#3600™ 6%, potato protein 2%, dried yeast 2%, CaCO₃ 0.5%, Adecanol LG-109 (deforming agent, Asahi Denka Co., Ltd.) 0.1% and Silicon KM-70 (deforming agent Shin-Etsu Chemical Co., Ltd.) 0.1% in 30-liter jar-fermenter. Production was carried out at 30° C. for 7 days under aeration of 20 liters/minute and agitation of 300 rpm.

### EXAMPLE 1

The culture broth (20 liters) obtained in Preparation 1 was extracted with 40 liters of methanol by intermittent mixing. The methanol extracted was filtered with an aid of diatomaceous earth and 60 liters of water was added. The mixture was passed through a column (10 liters) of Diaion HP-20 (Mitsubishi Chemical Co., Ltd.). The column was washed with 60% aqueous methanol (2 vol.) and eluted with 75% aqueous methanol (8 vol). This elute (75 liters) was concentrated in vacuo and substituted methanol solution. To the methanol solution (1.6 liters) was added ethyl acetate (8 liters) and obtained precipitate. The precipitate was dried in vacuo. The precipitate suspended in CH₂Cl₂ (25 vol) at 0° C. To the suspension was added Et₃SiH (10 eq.) and added dropwise trifluoroacetic acid (100 eq.). And then the solution was stirred for 2 to 2.5 hours at 25° C. The reaction mixture was slowly poured into pH 6.86 buffer (30 vol) at 0° C. adjusting to pH 8.5 to 10 with Na₂CO₃. After removing CH₂Cl₂ from the mixture in vacuo, chromatographed on a column of Sepabeads SP-205 (Mitsubishi Chemical Co., Ltd.). The column was washed with water (5 vol), 20% aqueous methanol (5 vol) and 40% aqueous methanol (5 vol) and then eluted with 60% aqueous methanol (5 vol) and 90% aqueous methanol (5 vol). The elute was concentrated in vacuo to an aqueous solution. To aqueous solution (600 ml) was added the fermentation broth (300 ml) of Streptomyces sp. No. 6907 obtained in Preparation 2 together with methanol (100 ml), and the reaction mixture was carried out at 37° C. for 3 hours. The reaction mixture was filtered with an aid diatomaceous earth. The filtrate was passed through a column of Diaion HP-20SS (Mitsubishi Chemical Co., Ltd.). The column was washed with 5% aqueous methanol (25 vol) and eluted with 10% aqueous methanol (20 vol). The fractions containing the object compound (1) was collected and evaporated in vacuo to give the object compound (1).

The Object Compound (1) of Example 1 as obtained has the following physico-chemical properties.

Appearance: white powder Nature: amphoteric substance Melting point: 250-260° C. (dec.) Specific rotation: [α]_{D}²³−15.0°(C: 0.5, H₂O) Molecular formula: C₃₅H₅₂N₈O₁₇S Molecular weight: ESI-MASS (m/z): 887 (M−H)⁻ Solubility: Soluble: water, dimethylformamide and dimethylsulfoxide Color reaction: Positive: iodine vapor reaction Ultraviolet absorption spectrum: λₘₐₓ^{water}: 277 nm IR (KBr): 3300, 2950, 1670, 1630, 1520, 1440, 1270, 1250, 1090, 1040, 970 cm⁻¹ ¹H-NMR (D₂O, 500 MHz) δ: 1.04 (3H, t, J=7 Hz), 1.24 (3H, d, J=6 Hz), 1.69-1.90 (3H, m), 2.10-2.30 (2H, m), 2.39 (1H, dd, J=9 and 15 Hz), 2.47 (1H, dd, J=4 and 15 Hz), 2.50-2.60 (2H, m), 2.67-2.78 (2H, m), 3.14 (1H, m), 3.47 (1H, dd, J=7 and 10 Hz), 3.55 (1H, m), 3.91-3.99 (2H, m), 4.65 (1H, dd, J=7 and 12 Hz), 4.72 (1H, m), 4.97-5.03 (2H, m), 6.97 (1H, d, J=8 Hz), 7.02 (1H, dd, J=2 and 8 Hz), 7.22 (1H, d, J=2 Hz) ¹³C-NMR (D₂O, 125 MHz) δ: 11.0 (q), 19.1 (q), 23.7 (t), 27.7 (d), 37.68 (t), 37.75 (d), 37.78 (t), 39.4 (t), 39.6 (t), 52.5 (t), 53.2 (d), 55.0 (d), 56.3 (t), 57.6 (d), 58.1 (d), 61.6 (d), 67.1 (d), 68.2 (d), 69.2 (d), 70.7 (d), 72.3 (d), 75.2 (d), 118.0 (d), 124.3 (d), 128.4 (d), 130.4 (s), 139.2 (s), 147.2 (s), 169.4 (s), 170.8 (s), 172.1 (s), 172.4 (s), 174.5 (s), 176.1 (s)

From the analysis of the above physical and chemical properties, and the result of the further investigation of identification of chemical structure, the chemical structure of the Object Compound (1) has been identified and assigned as follows.

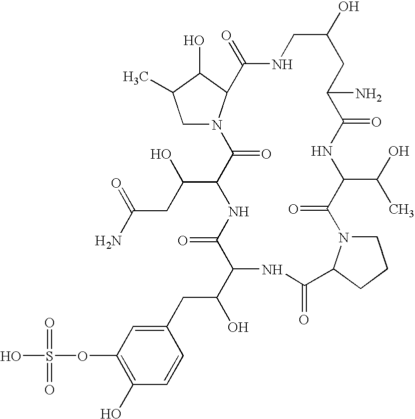

### EXAMPLE 2

The culture broth (20 liters) obtained in Preparation 1 was extracted with 40 liters of methanol by intermittent mixing. The methanol extracted was filtered with an aid of diatomaceous earth and 40 liters of water was added. The mixture was passed through a column (10 liters) of Diaion HP-20 (Mitsubishi Chemical Co., Ltd.). The column was washed with 60% aqueous methanol and eluted with 75% aqueous methanol. This elute (75 liters) was concentrated in vacuo to an aqueous solution (2.5 liters). To an aqueous solution was added 700 ml of the fermentation broth of Streptomyces sp. No. 6907 obtained in Preparation 2 together with 250 ml of methanol, and the reaction was carried out at 37° C. for 6 hours. The reaction was stopped with 78% sulfuric acid, and then pH was adjusted to 3.5. The reaction mixture was filtered with an aid of diatomaceous earth. The filtrate (3.4 liters) was concentrated in vacuo to 2 liters, and then the solution was passed through a column (6 liters) of Sepabeads SP-207 (Mitsubishi Chemical Co., Ltd.). The column was washed with 5% aqueous methanol and 15% aqueous methanol, and then washed with 30% aqueous methanol. The elute (30 liters) was concentrated in vacuo to an aqueous solution (5 liters), and then subjected to a column (1 liter) of YMC gel (ODS-AM 120 S-50, YMC Co., Ltd.). The column was washed with 3% aqueous methanol containing 0.05% phosphoric acid and eluted with 5% methanol containing 0.05% phosphoric acid. The elute (3 liters) was concentrated in vacuo to an aqueous solution (1.2 liters), and then applied to a column of YMC gel (ODS-AM 120 S-50, YMC Co., Ltd.). After washing with water, the active fraction was eluted with 80% aqueous methanol. The elute (1.1 liters) was concentrated in vacuo to an aqueous solution and lyophilized to give the Object Compound (2) (80 mg) as colorless powder.

The Object Compound (2) of Example 2 as obtained has the following physico-chemical properties.

Appearance: white powder Nature: amphoteric substance Melting point: 215-220° C. (dec.) Specific rotation: [α]_{D}²³−5.0°(C: 0.5, H₂O) Molecular formula: C₃₅H₅₂N₈O₂₀S Elemental Analysis: Calcd.: for C₃₅H₅₂N₈O₂₀S.11H₂O C, 37.04; H, 6.57; N, 9.87; (%) Found: C, 37.12; H, 6.06; N, 9.94; (%) Molecular weight: ESI-MASS (m/z): 935 (M−H)⁻ Solubility: Soluble: methanol, water Slightly soluble: ethanol Insoluble: ethyl acetate, acetone Color reaction: Positive: iodine vapor reaction, cerium sulfate reaction, Ninhydrin reaction Ultraviolet absorption spectrum: λₘₐₓ^{methanol} (E_{1 cm}^{1 %}): 210, 280 nm ¹H-NMR (D₂O, 500 MHz) δ: 1.02 (3H, t, J=7 Hz), 1.23 (3H, d, J=6 Hz), 2.00 (1H, m), 2.32-2.45 (3H, m), 2.57 (1H, m), 2.76 (2H, m), 3.46 (1H, m), 3.89-3.95 (2H, m), 4.05-4.13 (2H, m), 4.19-4.30 (3H, m), 4.36-4.50 (6H, m), 4.58 (1H, m), 4.93 (1H, d, J=6 Hz), 5.02 (1H, d, J=4 Hz), 5.40 (1H, d, J=3 Hz), 6.98 (1H, d, J=8 Hz), 7.03 (1H, dd, J=2 and 8 Hz), 7.26 (1H, d, J=2 Hz) ¹³C-NMR (D₂O, 125 MHz) δ: 11.2 (q), 19.2 (q), 31.6 (t), 37.7 (d), 39.4 (t), 39.7 (t), 52.7 (t), 53.2 (d), 53.3 (t), 54.8 (d), 57.5 (d), 57.8 (d), 65.0 (d)-, 67.2 (d), 67.3 (d), 69.1 (d), 70.7 (d), 71.6 (d), 72.4 (d), 74.7 (d), 75.2 (d), 75.8 (d), 118.0 (d), 124.3 (d), 128.6 (d), 130.5 (s), 139.3 (s), 147.3 (s), 169.2 (s), 169.8 (s), 172.2 (s), 172.3 (s), 173.2 (s), 176.1 (s)

From the analysis of the above physical and chemical properties, and the result of the further investigation of identification of chemical structure, the chemical structure of the Object Compound (2) has been identified and assigned as follows.

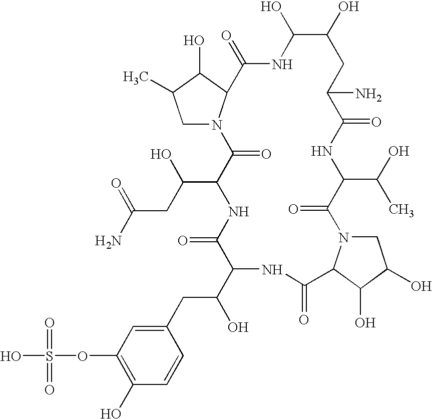

### EXAMPLE 3

The culture broth (20 liters) obtained in Preparation 1 was extracted with 40 liters of methanol by intermittent mixing. The methanol extracted was filtered with an aid of diatomaceous earth and 60 liters of water was added. The mixture was passed through a column (10 liters) of Diaion HP-20 (Mitsubishi Chemical Co., Ltd.). The column was washed with 60% aqueous methanol and eluted with 75% aqueous methanol (8 vol). This elute (75 liters) was concentrated in vacuo and substituted methanol solution. To the methanol solution (1.6 liters) was added ethyl acetate (8 liters) and obtained precipitate. The precipitate was dried in vacuo. The precipitate suspended in CH₂Cl₂ (25 vol) at 0° C. To the suspension was added ET₃SiH (10 eq.) and added dropwise trifluoroacetic acid (100 eq.). And then the solution was stirred for 2 to 2.5 hours at 25° C. The reaction mixture was slowly poured into pH 6.86 buffer (30 vol) at 0° C. adjusting to pH 8.5 to 10 with Na₂CO₃. After removing CH₂Cl₂ from the mixture in vacuo, chromatographed on a column of Sepabeads SP-205 (Mitsubishi Chemical Co., Ltd.). The column was washed with water (5 vol), 20% aqueous methanol (5 vol) and 40% aqueous methanol (5 vol). The elute was concentrated in vacuo to an aqueous solution. To aqueous solution (600 ml) was added the fermentation broth (300 ml) of Streptomyces sp. No. 6907 together obtained in Preparation 2 with methanol (100 ml), and the reaction mixture was carried out at 37° C. for 3 hours. The reaction mixture was filtered with an aid diatomaceous earth. The filtrate was passed through a column of Diaion HP-20SS (Mitsubishi Chemical Co., Ltd.). The column was washed with water (5 vol) and eluted with 5% qqueous methanol (25 vol). The fractions containing the Object Compound (3) was collected and evaporated in vacuo to give the object compound (3).

The Object Compound (3) of Example 3 as obtained has the following physico-chemical properties.

Appearance: white powder Nature: amphoteric substance Melting point: 255-260° C. (dec.) Specific rotation: [α]_{D}²³−14.0° (C: 0.5, H₂O) Molecular formula: C₃₄H₅₀N₈O₁₇S Molecular weight: ESI-MASS (m/z): 873 (M−H)⁻ Solubility: Soluble: water, dimethylformamide and dimethylsulfoxide Color reaction: Positive: iodine vapor reaction Ultraviolet absorption spectrum: λₘₐₓ^{water}: 277 nm IR (KBr): 3300, 2960, 1680, 1630, 1510, 1450, 1270, 1240, 960 cm⁻¹ ¹H-NMR (D₂O, 500 MHz) δ: 1.04 (3H, t, J=7 Hz), 1.68-1.87 (3H, m), 2.11-2.25 (2H, m), 2.10-2.30 (2H, m), 2.39 (1H, dd, J=9 and 15 Hz), 2.45-2.60 (3H, m), 2.67-2.80 (2H, m), 3.15 (1H, m), 3.44-3.55 (2H, m), 3.87-4.00 (4H, m), 4.05 (1H, dd, J=4 and 8 Hz), 4.21 (1H, dd, J=5 and 12 Hz), 4.27-4.33 (2H, m), 4.42 (1H, br d, J=2 Hz), 4.46 (1H, m), 4.52 (1H, m), 4.67 (1H, dd, J=7 and 11 Hz), 4.72 (1H, m), 4.99-5.03 (2H, m), 6.97 (1H, d, J=8 Hz), 7.02 (1H, dd, J=2 and 8 Hz), 7.21 (1H, d, J=2 Hz) ¹³C-NMR (D₂O, 125 MHz) δ: 11.0 (q), 23.8 (t), 27.9 (t), 37.7 (d), 37.7 (t), 38.1 (t), 39.4 (t), 39.5 (t), 52.5 (t), 53.5 (d), 55.0 (d), 55.2 (d), 56.2 (t), 57.8 (d), 61.5 (d), 61.6 (t), 68.3 (d), 69.4 (d), 70.7 (d), 72.3 (d), 75.2 (d), 118.0 (d), 124.3 (d), 128.4 (d), 130.4 (s), 139.2 (s), 147.2 (s), 169.4 (s), 170.4 (s), 171.5 (s), 172.1 (s), 172.4 (s), 174.6 (s), 176.2 (s)

From the analysis of the above physical and chemical properties, and the result of the further investigation of identification of chemical structure, the chemical structure of the Object Compound (3) has been identified and assigned as follows.

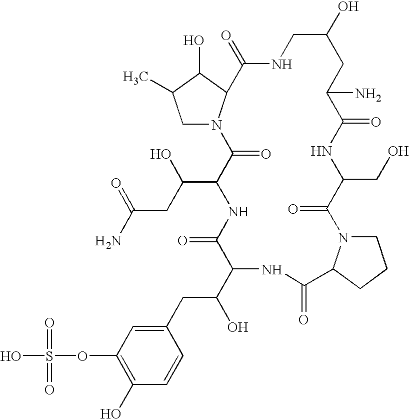

### EXAMPLE 4, 5, 6 and 7

The culture broth (20 ↓) obtained in Preparation 1 was extracted with methanol (40 ↓) by mixing. The methanol extract was filtered with an aid diatomaceous earth and water was added (60 ↓). The mixture was passed through a column of Diaion HP-20 (Mitsubishi Chemical Co., Ltd.). The column was washed with 60% aqueous methanol (2 vol) and eluted with 75% aqueous methanol (8 vol). This eluate was concentrated in vacuo to an aqueous. To aqueous solution (600 ml) was added the fermentation broth (300 ml) of Streptomyces sp. No.6907 obtained in Preparation 2 together with methanol (100 ml), and the reaction was carried out at 37° C. for 3 hours. The reaction mixture was filtered with an aid diatomaceous earth. The filtrate was passed through a column of Diaion HP-20 (Mitsubishi Chemical Co., Ltd.). The column was eluted with water (5 vol). The fractions containing the desired compound was collected and passed through a column of SP-207 (Mitsubishi Chemical Co., Ltd.). The column was washed with 5% aqueous methanol (5 vol), and eluted with 10% aqueous methanol (5 vol). This eluate was concentrated in vacuo to an aqueous. This aqueous solution was passed through a column of Daisopak C18 120BP (20Φ×250 mm; Daiso Co., Ltd.) and eluted with 3% aqueous methanol contains 0.1% H₃PO₄. (Retention time of Object Compound (4), Object Compound (5), Object Compound (6) and Object Compound (7) were 25.3 min, 29.8 min, 35.0 min and 40.6 min respectively.) The fractions containing the desired compound were collected and evaporated in vacuo. The concentrated solution was passed through a column of Daisopak C18 120BP (20Φ×250 mm; Daiso Co., Ltd.). The column was washed with water and eluted with 3% aqueous solution. The eluate was evaporated in vacuo. After all, Object Compound (4), Object Compound (5), Object Compound (6) and Object Compound (7) were obtained each.

The Object Compound (4) of Example 4 as obtained has the following physico-chemical properties.

Appearance: white powder Melting point: 170-180° C. (dec.) Specific rotation: [α]_{D}(²³° C.) −38° (C: 0.5, H₂O) Molecular formula: C₃₅H₅₂N₈O₁₇ Molecular weight: Molecular weight: 856.83 ESI-MASS (m/z): 857 (M+H)⁺, ESI-MASS (m/z): 855 (M−H)⁻ Solubility: Soluble: water, dimethylformamide and dimethylsulfoxide Infrared spectrum: νmax (KBr): 3350, 2980, 2940, 1660, 1630, 1530, 1450, 1280, 1240, 1120, 1080 cm⁻¹ ¹H-NMR (D₂O, 500 MHz) δ: 6.91 (1H, d, J=8 Hz), 6.84 (1H, d, J=2 Hz), 6.71 (1H, dd, J=8 and 2 Hz), 5.39 (1H, d, J=3 Hz), 5.04 (1H, br d, J=3 Hz), 4.91 (1H, d, J=6 Hz), 4.72 (1H, m), 4.62 (1H, m), 4.53 (1H, dd, J=12 and 17 Hz), 4.44-4.35 (4H, m), 4.26 (1H, d, J=5 Hz), 4.21-4.17 (2H, m), 4.10-4.04 (2H, m), 3.93-3.87 (2H, m), 3.38 (1H, dd, J=10 and 6 Hz), 2.58-2.31 (5H, m), 2.14 (1H, m), 2.00 (1H, m), 1.24 (3H, d, J=6 Hz), 0.62 (3H, d, J=7 Hz) ¹³C-NMR (D₂O, 125 MHz) δ: 176.2 (s), 173.8 (s), 172.3 (s), 172.2 (s), 172.1 (s), 170.0 (s), 169.1 (s), 145.0 (s), 144.8 (s), 132.4 (s), 120.5 (d), 116.9 (d), 115.3 (d), 75.9 (d), 75.3 (d), 75.1 (d), 74.6 (d), 70.8 (d), 70.8 (d), 69.2 (d), 67.2 (d), 61.7 (d), 58.0 (d), 56.2 (t), 55.6 (d), 54.8 (d), 53.2 (d), 52.7 (t), 39.7 (t), 37.6 (d), 37.4 (t), 31.7 (t), 19.2 (q), 11.2 (q)

From the analysis of the above physical and chemical properties, and the result of the further investigation of identification of chemical structure, the chemical structure of the Object Compound (4) has been identified and assigned as follows.

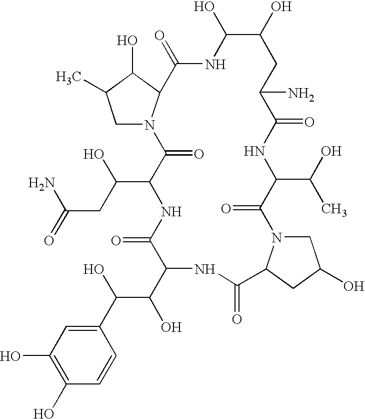

The Object Compound (5) of Example 5 as obtained has the following physico-chemical properties.

Appearance: white powder Melting point: 190-200° C. (dec.) Specific rotation: [α]_{D}(23° C.) −21°(C: 0.5, H₂O) Molecular formula: C₃₅H₅₂N₈O₂S₁ Molecular weight: Molecular weight: 952.89 ESI-MASS (m/z): 951 (M−H)⁻ Solubility: Soluble: water, dimethylformamide and dimethylsulfoxide Infrared spectrum: νmax (KBr): 3370, 2940, 1670, 1630, 1540, 1520, 1440, 1270, 1240, 1120, 1080, 1050 cm⁻¹ ¹³H-NMR (D₂O, 500 MHz) δ: 7.28 (1H, d, J=2 Hz), 7.12 (1H, dd, J=8 and 2 Hz), 7.04 (1H, d, J=8 Hz), 5.37 (1H, d, J=3 Hz), 5.01 (1H, br d, J=3 Hz), 4.96 (1H, d, J=5 Hz), 4.72 (1H, m), 4.65-4.53 (3H, m), 4.48-4.42 (3H, m), 4.35 (1H, d, J=4 Hz), 4.23-4.20 (2H, m), 4.08-4.03 (2H, m), 3.98-3.90 (2H, m), 3.83 (1H, dd, J=l1 and 7 Hz), 3.64 (1H, dd, J=11 and 7 Hz), 3.58 (1H, dd, J=11 and 8 Hz), 2.69 (1H, m), 2.55-2.30 (4H, m), 2.13 (1H, m), 2.01 (1H, m), 1.25 (3H, d, J=6 Hz) ¹³C-NMR (D₂O, 125 MHz) δ: 176.2 (s), 174.0 (s), 172.2 (s), 171.8 (s), 171.7 (s), 169.7 (s), 169.3 (s), 148.9 (s), 139.4 (s), 132.3 (s), 126.1 (d), 122.5 (d), 118.3 (d), 75.9 (d), 75.0 (d), 74.9 (d), 72.7 (d), 70.8 (d), 70.7 (d), 69.2 (d), 68:5 (d), 67.1 (d), 61.7 (d), 59.0 (t), 58.0 (d), 56.2 (t), 56.0 (d), 54.9 (d), 53.1 (d), 48.7 (t), 45.2 (d), 39.7 (t), 37.5 (t), 31.4 (t), 19.3 (q)

From the analysis of the above physical and chemical properties, and the result of the further investigation of identification of chemical structure, the chemical structure of the Object Compound (5) has been identified and assigned as follows.

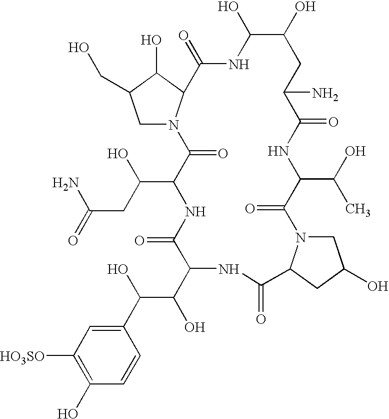

The Object Compound (6) of Example 6 as obtained has the following physico-chemical properties.

Appearance: white powder Melting point: 160-170° C. (dec.) Specific rotation: [α]_{D}(²³° C.) −15° (C: 0.5, H₂O) Molecular formula: C₃₅H₅₂N₈O₂S₁ Molecular weight: Molecular weight: 952.89 ESI-MASS (m/z): 951 (M−H)⁻ Solubility: Soluble: water, dimethylformamide and dimethylsulfoxide Infrared spectrum: νmax (KBr): 3360, 2980, 2940, 1670, 1630, 1520, 1440, 1270, 1080, 1050 cm⁻¹ ¹H-NMR (D₂O, 500 MHz) δ: 7.37 (1H, d, J=2 Hz), 7.14 (1H, dd, J=8 and 2 Hz), 7.05 (1H, d, J=8 Hz), 5.37 (1H, d, J=3 Hz), 4.98 (1H, br d, J=3 Hz), 4.92 (1H, d, J=6 Hz), 4.55 (1H, m), 4.52 (1H, d, J=8 Hz), 4.46-4.36 (5H, m), 4.28 (1H, d, J=5 Hz), 4.25-4.17 (3H, m), 4.12-4.04 (2H, m), 3.96-3.88 (2H, m), 3.41 (1H, dd, J=10 and 7 Hz), 2.56 (1H, m), 2.46-2.29 (3H, m), 1.99 (1H, m), 1.25 (3H, d, J=6 Hz), 1.00 (3H, d, J=7 Hz) ¹³C-NMR (D₂O, 125 MHz) δ: 176.2 (s), 172.8 (s), 172.3 (s), 172.2 (s), 171.8 (s), 169.8 (s), 169.3 (s), 148.9 (s), 139.5 (s), 132.5 (s), 126.4 (d), 122.3 (d), 118.3 (d), 76.0 (d), 74.97 (d), 74.95 (d), 74.95 (d), 74.6 (d), 71.6 (d), 70.8 (d), 69.1 (d), 67.3 (d), 67.1 (d), 64.9 (d), 57.5 (d), 56.5 (d), 54.8 (d), 53.3 (t), 53.2 (d), 52.7 (t), 39.7 (t), 37.7 (d), 31.5 (t), 19.3 (q), 11.2 (q)

From the analysis of the above physical and chemical properties, and the result of the further investigation of identification of chemical structure, the chemical structure of the Object Compound (6) has been identified and assigned as follows.

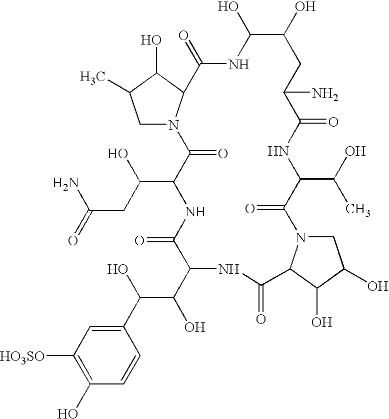

The Object Compound (7) of Example 7 as obtained has the following physico-chemical properties.

Appearance: white powder Melting point: 150-155° C. (dec.) Specific rotation: [α]_{D}(²³° C.) −29°(C: 0.5, H₂O) Molecular formula: C₃₄H₅₀N₈O₂₀S₁ Molecular weight: Molecular weight: 922.88 ESI-MASS (m/z): 921 (M−H)⁻ Solubility: Soluble: water, dimethylformamide and dimethylsulfoxide Infrared spectrum: νmax (KBr): 3360, 2940, 1670, 1630, 1520, 1450, 1270, 1240, 1080, 1050 cm⁻¹ ¹H-NMR (D₂O, 500 MHz) δ: 7.28 (1H, d, J=2 Hz), 7.12 (1H, dd, J=8 and 2 Hz), 7.04 (1H, d, J=8 Hz), 5.35 (1H, d, J=4 Hz), 5.03 (1H, m), 4.91 (1H, d, J=6 Hz), 4.72 (1H, m), 4.57 (1H, dd, J=11 and 7 Hz), 4.49 (1H, d, J=8 Hz), 4.43-4.33 (3H, m), 4.30 (1H, d, J=5 Hz), 4.25 (1H, m), 4.20 (1H, br d, J=2 Hz), 4.16 (1H, dd, J=11 and 4 Hz), 4.07-4.00 (2H, m), 3.95-3.85 (3H, m), 3.38 (1H, dd, J=10 and 7 Hz), 2.60-2.34 (5H, m), 2.12 (1H, m), 2.01 (1H, m), 1.0 (3H, d, J=7 Hz) ¹³C-NMR (D₂O, 125 MHz) δ: 176.2 (s), 174.0 (s), 172.2 (s), 172.0 (s), 171.6 (s), 169.3 (s), 169.2 (s), 148.9 (s), 139.4 (s), 132.3 (s), 126.0 (d), 122.4 (d), 118.3 (s), 76.3 (d), 74.94 (d), 74.91 (d), 74.5 (d), 70.8 (d), 70.7 (d), 69.5 (d), 67.2 (d), 61.6 (d), 61.6 (t), 56.1 (t), 56.1 (d), 55.3 (d), 54.9 (d), 53.3 (d), 52.7 (t), 39.6 (t), 37.8 (d), 37.5 (t), 31.7 (t), 11.2 (q)

From the analysis of the above physical and chemical properties, and the result of the further investigation of identification of chemical structure, the chemical structure of the Object Compound (7) has been identified and assigned as follows.

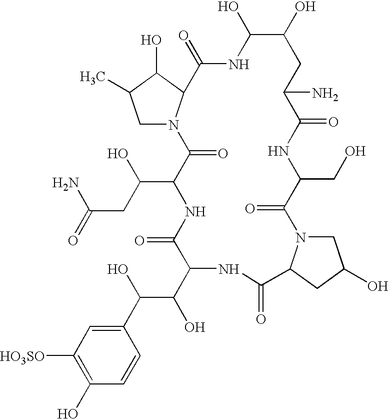

### EXAMPLE 8

To a solution of the Object Compound (1) of Example 1 (155 mg) and 4-[5-(4-pentyloxyphenyl)isoxazol-3-yl]benzoic acid benzotriazol-1-yl ester (89.9 mg) in N,N-dimethylformamide (1.5 ml) was added diisopropylethylamine (0.046 ml) and stirred for 6 hours at ambient temperature. The reaction mixture was pulverized with ethyl acetate. The precipitate was collected by filtration, and dried under reduced pressure. The powder was dissolved in pH 6.86 buffer and subjected to column chromatography on ODS (YMC-gel.ODS-AM.S-50 (Trademark: prepared by Yamamura Chemical Lab.)) eluting with 35% acetonitrile in water. The fractions containing the Object Compound (8) were combined and evaporated under reduced pressure to remove acetonitrile. The residue was lyophilized to give the Object Compound (8) (185 mg).

IR (KBr): 3365.2, 1658.5, 1635.3, 1257.4, 1045.2 cm⁻¹ MASS (m/z): 1220 (M−Na)⁻

From the analysis of the above physical and chemical properties, and the result of the further investigation of identification of chemical structure, the chemical structure of the Object Compound-(8) has been identified and assigned as follows.

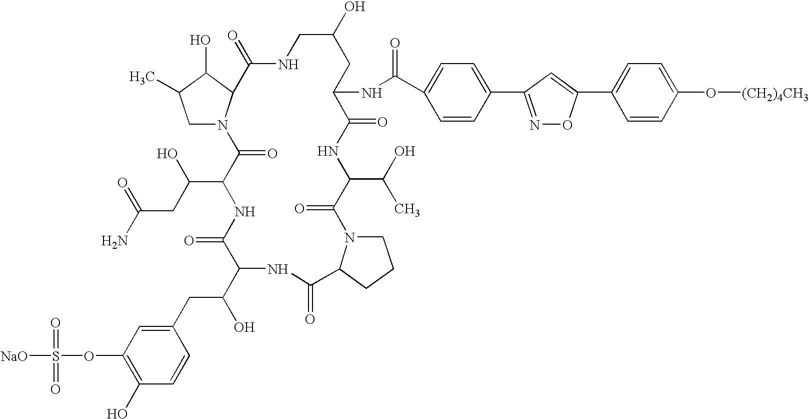

### EXAMPLE 9

To a solution of the Object Compound (2) of Example 2 (400 mg) and 4-[5-(4-pentyloxyphenyl)isoxazol-3-yl]benzoic acid benzotriazol-1-yl ester (220 mg) in N,N-dimethylformamide (4 ml) was added diisopropylethylamine (0.112 ml) and stirred for 7 hours at ambient temperature. The reaction mixture was pulverized with ethyl acetate. The precipitate was collected by filtration, and dried under reduced pressure. The powder was dissolved in water, and subjected to column chromatography on ion exchange resin (DOWEX-50WX4 (Trademark: prepared by Dow Chemical)) eluting with water. The fractions containing the Object Compound (9) were combined and subjected to column chromatography on ODS (YMC-gel.ODS-AM.S-50 (Trademark: prepared by Yamamura Chemical Lab.)) eluting with 18% acetonitrile in water. The fractions containing the Object Compound (9) were combined, and evaporated under reduced pressure to remove acetonitrile. The residue was lyophilized to give the Object Compound (9) (309 mg).

IR (KBr): 3347.8, 1664.3, 1627.6, 1508.1, 1438.6, 1257.4, 1047.2 cm⁻¹ NMR (DMSO-d₆, δ): 0.91 (3H, d, J=7.1 Hz), 0.98 (3H, d, J=6.7 Hz), 1.07 (3H, d, J=5.9 Hz), 1.2-1.5 (4H, m), 1.6-2.6 (6H, m), 3.20 (1H, m), 3.6-4.5 (17H, m), 4.6-5.6 (12H, m), 6.71 (1H, d, J=8.1 Hz), 6.79 (1H, s), 6.82 (1H, d, J=8.1 Hz), 7.10 (1H, s), 7.12 (2H, d, J=8.9 Hz), 7.2-7.6 (4H, m), 7.85 (2H, d, J=8.9 Hz), 7.9-8.2 (5H, m), 8.35 (1H, d, J=8.8 Hz), 8.73 (1H, m), 8.89 (1H, d, J=7.9 Hz) MASS (m/z): 1268 (M−Na)⁻ Elemental Analysis: Calcd.: for C₅₆H₇₀N₉O₂₃S.8H₂O C, 46.83; H, 6.03; N, 8.78; (%) Found: C, 46.79; H, 5.96; N, 8.74; (%)

From the analysis of the above physical and chemical properties, and the result of the further investigation of identification of chemical structure, the chemical structure of the Object Compound (9) has been identified and assigned as follows.

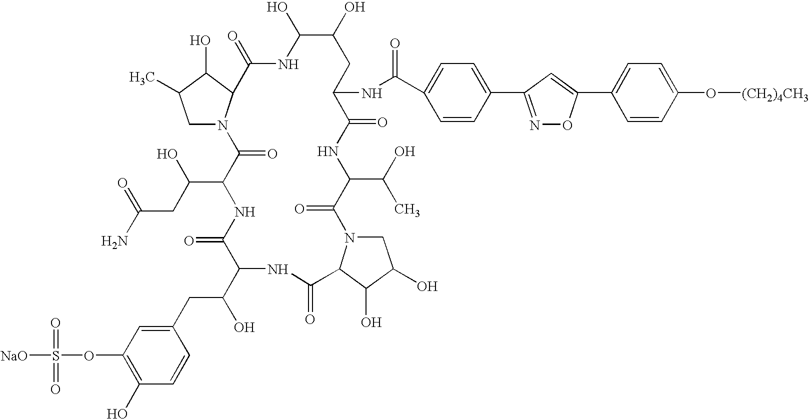

### EXAMPLE 10

To a solution of the Object Compound (3) of Example 3 (150 mg) and 4-[5-(4-pentyloxyphenyl)isoxazol-3-yl]benzoic acid benzotriazol-l-yl ester (88.4 mg) in N,N-dimethylformamide (1.5 ml) was added diisopropylethylamine (0.045 ml) and stirred for 6 hours at ambient temperature. The reaction mixture was pulverized with ethyl acetate. The precipitate was collected by filtration, and dried under. reduced pressure. The powder was dissolved in pH 6.86 buffer and subjected to column chromatography on ODS (YMC-gel.ODS-AM.S-50 (Trademark: prepared by Yamamura Chemical Lab.)) eluting with 35% acetonitrile in water. The fractions containing the Object Compound (10) were combined and evaporated under reduced pressure to remove acetonitrile. The residue was lyophilized to give the Object Compound (10) (160 mg).

IR (KBr): 3365.2, 1664.3, 1635.3, 1257.4, 1045.2 cm⁻¹ MASS (m/z): 1206 (M−Na)⁻

From the analysis of the above physical and chemical properties, and the result of the further investigation of identification of chemical structure, the chemical structure of the Object Compound (10) has been identified and assigned as follows.

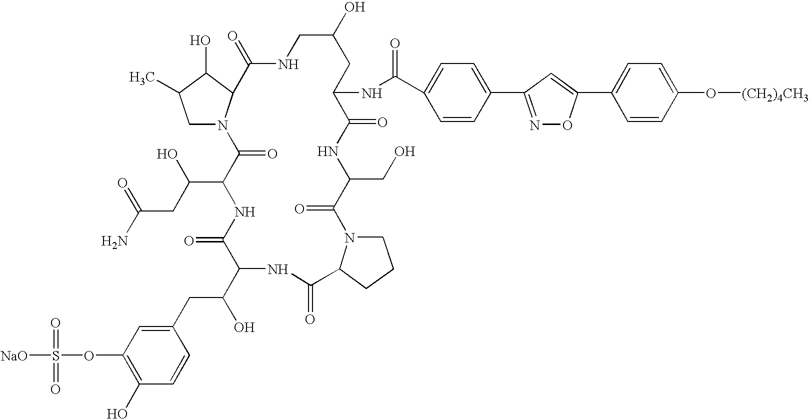

1. A polypeptide compound of the following general formula (I): 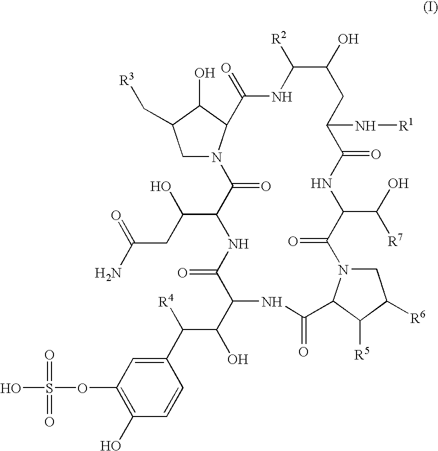
wherein R¹ is hydrogen or acyl group,
R², R³, R⁴, R⁵ and R⁶ are each independently hydrogen or hydroxy, and
R⁷ is hydrogen or lower alkyl,
or a salt thereof.

2. A process for preparing a polypeptide compound of the following general formula (I): 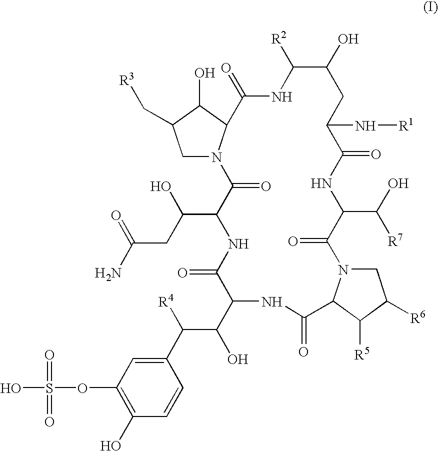
wherein R¹ is hydrogen or acyl group,
R², R³, R⁴, R⁵ and R⁶ are each independently hydrogen or hydroxy, and
R⁷ is hydrogen or lower alkyl,
or a salt thereof, which comprises
i) fermenting a strain belonging to the genus Coleophoma which is capable of producing a compound of the formula (Ia); or a salt thereof: 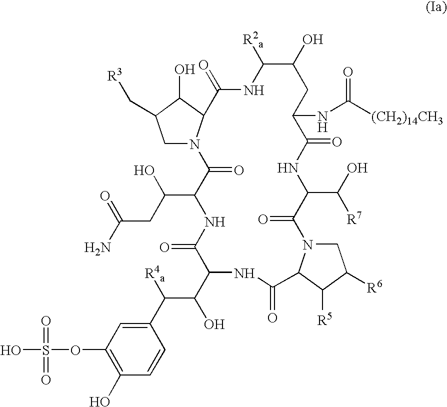
wherein R³, R⁵ and R⁶ are each independently hydrogen or hydroxy,
Rₐ² and Rₐ⁴ are hydroxy, and
R⁷ is hydrogen or lower alkyl,
in a nutrient medium and recovering the compound (Ia) or a salt thereof, to give the compound (Ia) or a salt thereof, or
ii) reducing a compound of (Ia) or a salt thereof, to give a compound of the formula (Ib): 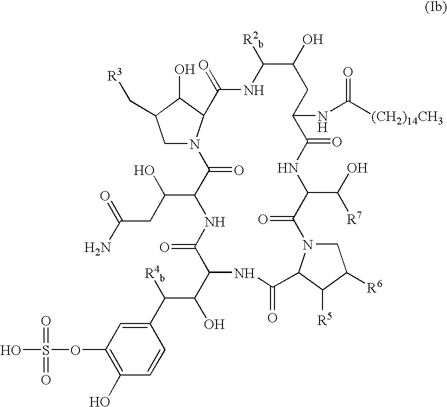
wherein R³, R⁵, R⁶ and R⁷ are defined above, and
R_{b}² and R_{b}⁴ are hydrogen,
or a salt thereof,
iii) subjecting a compound (Ic): 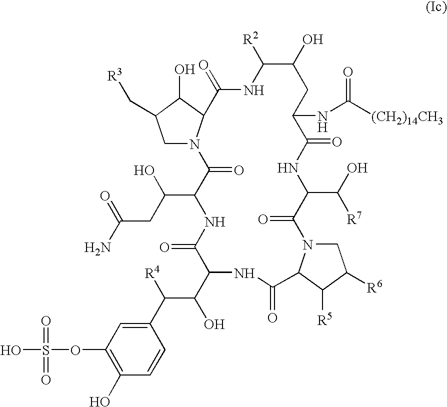
wherein R³, R⁵, R⁶ and R⁷ are defined above, and
R² and R⁴ are independently hydrogen or hydroxy,
or a salt thereof, to elimination reaction of N-acyl group, to give a compound of the formula (Id): 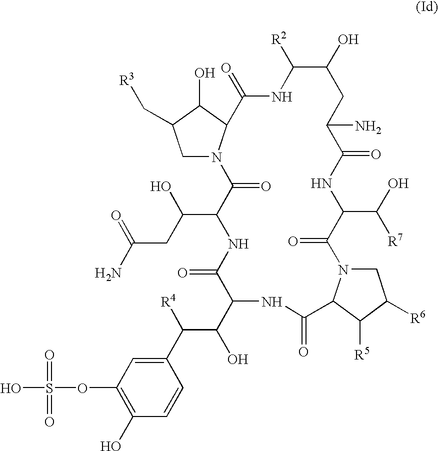
wherein R², R³, R⁴, R⁵, R⁶ and R⁷ are defined above,
or a salt thereof, or
iv) reacting a compound of (Id) or a salt thereof, with a compound (II) of the formula:
Rₐ¹—OH (II)
or its reactive derivative at the carboxy group
or a salt thereof, to give a compound of the formula (Ie): 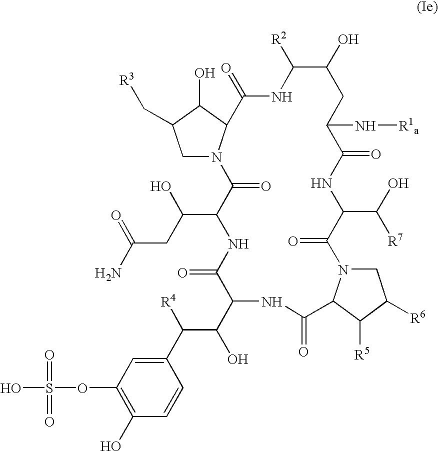
wherein R², R³, R⁴, R⁵, R⁶ and R⁷ are defined above, and
Rₐ¹ is acyl group exclusive of palmitoyl,
or a salt thereof.

3. A pharmaceutical composition which comprises, as an active ingredient, a compound of claim 1 or a pharmaceutically acceptable salt thereof in admixture with pharmaceutically acceptable carriers or excipients.

4. Use of a compound of claim 1 or a pharmaceutically acceptable salt thereof for the manufacture of a medicament.

5. A compound of claim 1 or a pharmaceutically acceptable salt thereof for use as a medicament.

6. A method for the prophylactic and/or the therepeutic treatment of infectious diseases caused by pathogenic microorganisms, which comprises administering a compound of claim 1 or a pharmaceutically acceptable salt thereof to a human being or an animal.

7. A commercial package comprising the pharmaceutical composition of claim 3 and a written matter associated therewith, wherein the written matter states that the pharmaceutical composition can or should be used for preventing or treating infectious disease.

8. An article of manufacture, comprising packaging material and the compound (I) identified in claim 1 contained within said packaging material, wherein said compound (I) is therapeutically effective for preventing or treating infectious diseases, and wherein said packaging material comprises a label or a written material which indicates that said compound (I) can or should be used for preventing or treating infectious diseases.
